(19) United States
(12) Patent Application Publication
(10) Pub. No.: US 2020/0179243 A1
(43) Pub. Date: Jun. 11, 2020

(54) TOPICAL COMPOSITIONS AND METHODS OF USING THEREOF

(71) Applicant: OHIO STATE INNOVATION FOUNDATION, Columbus, OH (US)

(72) Inventors: Prabir K. DUTTA, Worthington, OH (US); Bo WANG, Columbus, OH (US)

(73) Assignee: Ohio State Innovation Foundation, Columbus, OH (US)

(21) Appl. No.: 16/321,673

(22) PCT Filed: Jul. 31, 2017
(86) PCT No.: PCT/US2017/044733

§ 371 Date: Jan. 29, 2019

Related U.S. Application Data
(60) Provisional application No. 62/430,613, filed on Dec. 6, 2016.
(60) Provisional application No. 62/430,610, filed on Dec. 6, 2016.
(60) Provisional application No. 62/368,654, filed on Jul. 29, 2016.

(51) Int. Cl. A61K 8/02; A01N 37/44; A61K 8/26; A61Q 17/04; A61K 8/35; A61K 8/27; A61K 8/19

(52) U.S. Cl. CPC A61K 8/0279; A01N 37/44; A61K 8/26; A61K 8/19; A61K 8/35; A61K 8/27; A61Q 17/04

(57) ABSTRACT

Disclosed herein are topical compositions for the administration of active agents. The compositions can comprise zeolite nanoparticles dispersed in a topically acceptable carrier. The zeolite nanoparticles can further comprise an effective amount of an active agent adsorbed on the zeolite nanoparticles, encapsulated within the zeolite nanoparticles, or a combination thereof. Also provided sunscreen agents and antimicrobial agents, as well as compositions comprising sunscreen agents and antimicrobial agents.

### CROSS REFERENCE TO RELATED APPLICATIONS

This application claims the benefit of U.S. Provisional Patent Application Ser. No. 62/368,654, filed Jul. 29, 2016, U.S. Provisional Patent Application Ser. No. 62/430,610, filed Dec. 6, 2016, and U.S. Provisional Patent Application Ser. No. 62/430,613, filed Dec. 6, 2016, each of which is expressly incorporated herein by reference.

### BACKGROUND

The topical delivery of active agents remains an area of intense interest. While topical formulations for the administration of active agents are widely employed, many formulations suffer from significant shortcomings. For example, may topical formulations fail to provide for controlled or extended release of active agents upon application to a subject's skin. Further, many active agents are chemically and photochemically unstable and/or elicit allergic responses when applied to the skin of a subject. Improved compositions for topical administration offer the possibility to address these and other shortcomings.

### SUMMARY

Provided herein are topical compositions for the administration of active agents. The compositions can comprise zeolite nanoparticles dispersed in a topically acceptable carrier. The zeolite nanoparticles can further comprise an effective amount of an active agent adsorbed on the zeolite nanoparticles, encapsulated within the zeolite nanoparticles, or a combination thereof.

The zeolite nanoparticles can have an average particle size of less than 250 nm (e.g., less than 100 nm, such as from 10 to 100 nm or from 10 to 50 nm). The zeolite nanoparticles can exhibit an internal surface area of from 100 to 1,000 m²/g (e.g., an internal surface area of from 200 to 1,000 m²/g). In some cases, the zeolite nanoparticles can comprise a faujasite structure.

In some cases, the zeolite nanoparticles can be modified, for example, to alter the surface chemistry of the zeolite nanoparticles. Depending on the active agent, zeolite, and the intended use of the composition, modification of the zeolite nanoparticles can alter the release characteristics of the active agent (e.g., stimulate release of the active agent, ensure encapsulation of the active agent), improve the dispersability of the zeolite nanoparticles in the topically acceptable carrier, increase the affinity of the zeolite nanoparticles for a target, or a combination thereof.

In some cases, the zeolite nanoparticles can have a hydrophobic surface (e.g., a surface that is covalently modified to increase its hydrophobicity). In some cases, the zeolite nanoparticles can have a hydrophilic surface (e.g., a surface that is covalently modified to increase its hydrophilicity). In some cases, the zeolite nanoparticles can have a positively or negatively charged surface (e.g., a surface that is modified to increase the zeta potential of the zeolite nanoparticles). In other cases, the zeolite nanoparticles can be modified to have a neutral surface.

The active agent can be, for example, a UV-blocking agent, antimicrobial agent, insecticide, cosmetic agent, fragrance, anesthetic agent, keratolytic agent, steroid, anthelmintic agent, dermatological agent, antioxidant, anti-inflammatory agent, or combination thereof. The active agent can comprise, for example, metal nanoparticles, metal ions, small molecules (e.g., organic small molecules), or a combination thereof. In some embodiments, the active agent has a molecular size of 13 Angstroms or less. In certain embodiments, the active agent can comprise a hydrophilic small molecule, hydrophobic small molecule, a charged small molecule, a neutral small molecule, or a combination thereof). In some embodiments, the active agent can comprise an insecticide (e.g., N,N-diethyl-meta-toluamide (DEET)). In some embodiments, the active agent can be a UV-blocking agent (e.g., avobenzone, oxybenzone, or a combination thereof). In some embodiments, the active agent can be an antimicrobial agent (e.g., silver nanoparticles, silver ions, copper ions, zinc ions, or a combination thereof). The active agent can be present in an amount of from 1% to 25% by weight (e.g., from 5% to 20% by weight), based on the total weight of the zeolite nanoparticles.

In some embodiments, the active agent can be encapsulated within the zeolite nanoparticles. In certain embodiments, the active agent can be encapsulated within the zeolite nanoparticles, and the active agent remains encapsulated within the zeolite nanoparticles upon application of the composition to a subject's skin. In some embodiment, encapsulation of the active agent can stabilize the active agent against degradation (e.g., chemical degradation resulting from exposure to water, heat, sunlight, or a combination thereof) during storage and/or following application of the composition to a subject's skin. For example, in some cases, the active agent can be stable towards degradation for a period of at least eight hours upon application of the composition to the subject's skin. In another example, the active agent can be stable towards degradation for a period of at least six months when stored at room temperature in the absence of light. In some embodiments, encapsulation of the active agent can sequester the active agent from the subject, thereby minimizing and/or eliminating the subject's allergic response to the active agent. In some embodiments, encapsulation of the active agent can sequester the active agent from other components of the composition, thereby allowing, for example, two active agents that undesirably react with one another to be included in the same composition.

In some embodiments, the active agent is adsorbed on the zeolite nanoparticles, encapsulated within the zeolite nanoparticles, or a combination thereof, and the active agent can be released from the zeolite nanoparticles upon application of the composition to a subject's skin. In some embodiments, the zeolite nanoparticle can provide for the extended release of the active agent upon application of the composition to a subject's skin. For example, in some cases, the active agent can be released over an extended period of time (e.g., over a period of at least four hours) following application of the composition to a subject's skin.

Also provided herein are sunscreen agents, as well as compositions comprising these sunscreen agents dispersed in a topically acceptable carrier. The sunscreen agents can comprise an organic UV-blocking agent encapsulated within a porous inorganic nanomaterial. In some embodiments, the sunscreen agent can comprise from 5% to 20% by weight (e.g., from 10% to 20% by weight) UV-blocking agent, based on the total weight of the sunscreen agent.

The UV-blocking agent can be an organic compound that absorbs light in the UV region at one or more wavelengths from 290 nanometers (nm) to 400 nm. For example, the UV-blocking agent can exhibit a molar extinction coefficient of at least 10,000 mol⁻¹ L cm⁻¹ (e.g., at least 25,000 mol⁻¹ L cm⁻¹, at least 50,000 mol⁻¹ L cm⁻¹, at least 75,000 mol⁻¹ L cm⁻¹, or at least 100,000 mol⁻¹ L cm⁻¹) for at least one wavelength within the range of from 290 nm to 400 nm.

In some embodiments, the UV-blocking agent can be an organic compound that absorbs light in the UV-B region at one or more wavelengths from 290 nm to 320 nm (i.e., a UV-B blocking agent). For example, the UV-blocking agent can exhibit a molar extinction coefficient of at least 10,000 mol⁻¹ L cm⁻¹ (e.g., at least 25,000 mol⁻¹ L cm⁻¹, at least 50,000 mol⁻¹ L cm⁻¹, at least 75,000 mol⁻¹ L cm⁻¹, or at least 100,000 mol⁻¹ L cm⁻¹) for at least one wavelength within the range of from 290 nm to 320 nm. In some cases, the UV-blocking agent can exhibit a molar extinction coefficient of at least 10,000 mol⁻¹ L cm⁻¹ at all wavelengths within the range of from 290 nm to 320 nm.

In some embodiments, the UV-blocking agent can be an organic compound that absorbs light in the UV-A region at one or more wavelengths from 320 nm to 400 nm (i.e., a UV-A blocking agent). For example, the UV-blocking agent can exhibit a molar extinction coefficient of at least 10,000 mol⁻¹ L cm⁻¹ (e.g., at least 25,000 mol⁻¹ L cm⁻¹, at least 50,000 mol⁻¹ L cm⁻¹, at least 75,000 mol⁻¹ L cm⁻¹, or at least 100,000 mol⁻¹ L cm⁻¹) for at least one wavelength within the range of from 320 nm to 400 nm. In some cases, the UV-blocking agent can exhibit a molar extinction coefficient of at least 10,000 mol⁻¹ L cm⁻¹ at all wavelengths within the range of from 320 nm to 400 nm.

Examples of suitable UV-blocking agents include, for example, p-aminobenzoic acid, padiate O, phenylbenzimidazole sulfonic acid, cinoxate, dixoybenzone, oxybenzone, homosalate, menthyl anthranilate, octocrylene, octyl methoxycinnamate, octyl salicylate, sulisobenzone, trolamine salicylate, avobenzone, ecamsule, 4-methylbenzylidene camphor, bisoctrizole, bemotrizinol, bisdisulizole disodium, tris-biphenyl triazine, drometrizole trisiloxane, benzophenone-9, ethylhexyl triazone, diethylamino hydroxybenzoyl hexyl benzoate, iscotrizinol, polysilicone-15, amiloxate, and combinations thereof. In some embodiments, the UV-blocking agent can be p-aminobenzoic acid, padiate O, phenylbenzimidazole sulfonic acid, cinoxate, dixoybenzone, oxybenzone, homosalate, menthyl anthranilate, octocrylene, octyl methoxycinnamate, octyl salicylate, sulisobenzone, trolamine salicylate, avobenzone, ecamsule, or a combination thereof. In certain embodiments, the UV-blocking agent can be avobenzone, oxybenzone, or a combination thereof.

The porous inorganic nanomaterial can be nanoparticles formed from a microporous or mesoporous inorganic material. Preferably, the porous inorganic nanomaterial can be capable of scattering UV light. In some embodiments, the porous inorganic nanomaterial can have an average particle size of less than 250 nm (e.g., less than 100 nm). In certain cases, the porous inorganic nanomaterial has an average particle size of from 10 to 100 nm (e.g., from 10 to 50 nm). The porous inorganic nanomaterial can possesses a very regular pore structure of molecular dimensions. In some cases, the porous inorganic nanomaterial can exhibit a monodisperse pore size distribution. In certain embodiments, the porous inorganic nanomaterial can exhibit a pore size of from 10 to 75 angstroms (e.g., from 10 to 50 angstroms). The porous inorganic nanomaterial can also possess a high internal surface area. For example, in some embodiments, the porous inorganic nanomaterial can exhibit an internal surface area of from 100 to 1,000 m²/g (e.g., from 200 to 1,000 m²/g).

In some embodiments, the porous inorganic nanomaterial can comprise alumino-silicate nanoparticles (e.g., zeolite nanoparticles). In certain embodiments, the porous inorganic nanomaterial can comprise zeolite nanoparticles having a faujasite structure. In other embodiments, the porous inorganic nanomaterial comprises nanoparticles formed from a metal-organic framework. The metal-organic framework can be, for example, an iron(III) dicarboxylate framework, an iron(III) tetramethylterephthalate framework, an iron(III) muconate framework, a zinc terephthalate framework, a zinc imidazolate framework, or a combination thereof. Suitable metal organic frameworks are known in the art, and include, for example, metal-organic frameworks such as MIL-88A, MIL-88B-4CH3, MIL-89, MIL-100(Fe), MIL-53(Fe), MOF-5, ZIF-8, and combinations thereof.

In some embodiments, the porous inorganic nanomaterial can be hydrophobically modified. In certain embodiments, the porous inorganic nanomaterial can comprise alumino-silicate nanoparticles (e.g., zeolite nanoparticles) whose surfaces are covalently modified to increase their hydrophobicity. For example, the porous inorganic nanomaterial can comprise an alumino-silicate nanoparticle (e.g., a zeolite nanoparticle) whose surface has been covalently modified with a caprylylsilane (e.g., with a trialkoxycaprylylsilane such as trimethoxycaprylylsilane) to increase its hydrophobicity.

In some embodiments, the porous inorganic nanomaterial can further comprise a quenching species. The quenching species can comprise a quenching ion. For example, the quenching ion can be an ion introduced by ion exchange into the porous inorganic nanomaterial (e.g., into the zeolite nanoparticle). Examples of suitable quenching ions include cations, such as alkali metal ions, transition metal ions, rare earth ions, and combinations thereof. The quenching species can also be an organic molecule, such as nitromethane, an amine compound, or a combination thereof.

The sunscreen agent can be present in the composition in an amount of from 0.5% to 10% by weight, based on the total weight of the composition. The composition can be formulated to exhibit an SPF of at least 15 (e.g., at least 30), as measured using the international standard ISO 24444: 2010(E). The composition can be appropriately formulated for topical application to a subject (e.g., for application to the skin of a subject). For example, the composition can be a cream, dispersion, emulsion, gel, ointment, lotion, milk, mousse, spray, or tonic. In some embodiments, the composition can be a sunscreen or cosmetic.

Also provided herein are antimicrobial agents, compositions, and methods of using the antimicrobial agents. The antimicrobial agents comprise zeolite nanoparticles, wherein the zeolite nanoparticles comprise an effective amount of silver to kill or inhibit the growth of a microbe.

The zeolite nanoparticles are porous and the silver can be disposed within and/or on a surface of the zeolite nanoparticles. The average particle size of the zeolite nanoparticles can be 100 nm or less (e.g., 80 nm or less). In certain cases, the zeolite nanoparticles have an average particle size of from 10 to 100 nm (e.g., from 20 to 60 nm). The zeolite nanoparticles can possesses a very regular pore structure of molecular dimensions. In some cases, the zeolite nanoparticles can exhibit a monodisperse pore size distribution. In certain embodiments, the zeolite nanoparticles can exhibit an internal pore size of from 2 to 13 angstroms and/or an external pore size of from 10 to 75 angstroms (e.g., from 10 to 50 angstroms) due to packing of the nanoparticles. The zeolite nanoparticles can also possess a high internal surface area. For example, in some embodiments, the zeolite nanoparticles can exhibit an internal surface area of at least 150 m²/g (e.g. at least 200 m²/g, at least 300 m²/g, at least 350 m²/g, or from 300 to 700 m²/g). In some embodiments, the zeolite nanoparticles can have a faujasite structure.

In some embodiments, the silver present in the antimicrobial agents can comprise silver nanoparticles. In some cases, the silver nanoparticles consist of silver metal that have antimicrobial activity. The silver nanoparticles can have an average size of 10 nm or less (e.g., from 1 nm to 10 nm or from 1 nm to 5 nm). The amount of silver nanoparticles present in the antimicrobial agents can be 1% by weight or greater, based on the total weight of the zeolite nanoparticles and the silver. In some embodiments, the silver nanoparticles can be present in an amount from 1% to 25% by weight (e.g., from 1% to 20% by weight, from 5% to 25% by weight, from 5% to 20% by weight, from 10% to 25% by weight, from 10% to 20% by weight, or from 15% to 25% by weight), based on the total weight of the zeolite nanoparticles and the silver.

In some embodiments, the silver present in the antimicrobial agents can comprise silver ions. The silver ions may be retained at ion-exchangeable sites of the zeolite nanoparticles. The silver ions can be present in an amount of 10% or greater (e.g., from 10% up to 100%, from 10% to 95%, from 20% up to 100%, from 30% up to 100%, from 40% up to 100%, or from 50% up to 100%) of the ion exchange capacity of the zeolite nanoparticles.

The zeolite nanoparticles can further comprise an adjuvant. In some embodiments, the adjuvant includes antimicrobial metal ions retained at ion-exchangeable sites of the zeolite nanoparticles. The antimicrobial metal ions can include copper ions, zinc ions, or a combination thereof. In some embodiments, the adjuvant includes hydrogen ions. The hydrogen ions may be present in an effective amount to reduce the pH of a region (e.g., an aqueous region) in contact with the zeolite nanoparticles. In some embodiments, the adjuvant includes a small molecule antimicrobial agent. In some cases, the small molecule antimicrobial agent is hydrophilic. The small molecule antimicrobial agent can include an antibiotic, an antiseptic, or a disinfectant. The small molecule antimicrobial agent can be present in an amount of from 1% to 20% by weight, based on the total weight of the zeolite nanoparticles and the silver nanoparticle.

The zeolite nanoparticles can also comprise a microbial targeting agent. The microbial targeting agent can be covalently bound to a surface of the zeolite nanoparticles. In some embodiments, the microbial targeting agent can comprise a cationic group or a cationic precursor. For example, the microbial targeting agent can comprise an alkyl amine such as a C₁-C₆ amine.

Some exemplary embodiments of the antimicrobial agents disclosed herein can include zeolite nanoparticles, wherein the zeolite nanoparticles further comprise silver nanoparticles disposed on the zeolite nanoparticles and antimicrobial metal ions retained at ion-exchangeable sites within the zeolite nanoparticles. Other exemplary embodiments of the antimicrobial agents disclosed herein can include zeolite nanoparticles, wherein the zeolite nanoparticles further comprise silver nanoparticles disposed on the zeolite nanoparticles and wherein a surface of the zeolite nanoparticles is functionalized with a microbial targeting agent. Further exemplary embodiments of the antimicrobial agents disclosed herein can include zeolite nanoparticles, wherein the zeolite nanoparticles further comprise silver nanoparticles disposed on the zeolite nanoparticles and a small molecule antimicrobial agent adsorbed on and/or within the zeolite nanoparticle.

As discussed herein, compositions comprising the antimicrobial agents are also disclosed. In certain embodiments, the compositions can be in the form of a powder comprising the zeolite nanoparticles and silver. In certain embodiments, the compositions can be in the form of a dispersion comprising the zeolite nanoparticles and silver dispersed in a carrier. The carrier may depend on the application of the antimicrobial agent, however, in some embodiments, the carrier can include an aqueous or organic solvent.

Articles comprising the antimicrobial compositions disclosed herein are also provided. In some embodiments, the article can include the zeolite nanoparticles dispersed on a surface of the article, wherein the zeolite nanoparticles comprise an effective amount of silver to kill or inhibit the growth of a microbe. In some examples, the article can be a medical device, a food package, or a coating.

Also disclosed herein are methods of using the antimicrobial agents. In some embodiments, the antimicrobial agents can be used to kill or inhibit the growth of a microbe, the method comprising exposing the microbe to a composition comprising zeolite nanoparticles, wherein the zeolite nanoparticles comprise an effective amount of silver to kill or inhibit the growth of the microbe. In some embodiments, the antimicrobial agents can be used to treat or prevent a microbial infection in a subject, the method comprising administering a composition comprising zeolite nanoparticles to the subject, wherein the zeolite nanoparticles comprise a therapeutically effective amount of silver.

The microbe can be selected from a bacteria, a fungi, a virus, an algae, or a combination thereof. In some examples, the microbe can be a bacteria selected from Escherichia coli, Staphylococcus aureus, Bacillus coagulans, Bacillus megaterium, Bacillus subtilis, Enterococcus faecium, Pseudoxanthomonas spp Pseudomonas putida, Pseudomonas aeruginosa, Pseudomonas maculicola, Pseudomanas chlororaphis, Pseudomonas flourescens, Nocardia brasiliensis, Nocardia globerula, Acinetobacter genomospecies, Acinetobacter calcoaceticus, Acinetobacter baumannii, Stenotrophomonas maltophlia, Pantoea stewartii ss stewartii, Chryseobacterium balustinus, Duganella zoogloeoides, Chryseobacterium meningosepticum, Staphylococcus hominis, Nocardia transvalensis, Burkolderia glumea, Pediococcus acidilactici/parvulus, Sphingomonas terrae, Corynebacterium spp Gordonia rubripertincta, Rhodococcus rhodnii, Brevundimonas vesicularis, Providencian heimbachae, Gordonia sputi, Cellulosimicrobium cellulans, Sphingomonas sanguinis, Hydrogenophaga pseudoflava, Actinomadura cremea, Xanthomonas spp. or a combination thereof.

In some examples, the microbe can be a fungi selected from Candida albicans, Candida parapsilosis, Candida tropicalis, Candida glabrata, Kluyveromyces marxianus, Hyphopichia burtanii, Fusarium oxysporum, Botrytis cinerea, Aspergillus niger, Alternaria alternata, Sclerotinia sclerotiorum, Paecilomyces lilacinus, Penicillium vinaceum, Penicillium expansum, Penicillium charlesii, Penicillium expansum, or a combination thereof.

The microbe can be present on a surface of or in a food product, a wound, a medical device, a pharmaceutical product, a personal care product, an equipment, a wall, a liquid, or a combination thereof.

### DESCRIPTION OF DRAWINGS

FIG. 1 is a scheme illustrating the structure of avobenzone, and the makeup of three samples evaluated spectroscopically (avobenzone alone, avobenzone encapsulated in a zeolite nanoparticle, and avobenzone encapsulated in a zeolite nanoparticle that includes a quenching ion) (AB=avobenzone, NZ=nanozeolite).

FIG. 2 is a plot illustrating the normalized absorbance of each sample (avobenzone alone, square trace; avobenzone encapsulated in a zeolite nanoparticle, circle trace; and avobenzone encapsulated in a zeolite nanoparticle that includes a quenching ion, diamond trace) as a function of photolysis time. Encapsulation of avobenzone in the zeolite nanoparticle stabilizes the avobenzone to degradation.

FIG. 3 illustrates a UV/Vis absorbance spectrum of avobenzone alone (bottom trace) compared with a UV/Vis absorbance spectrum of avobenzone encapsulated in a zeolite nanoparticle (top trace). Upon encapsulation of the avobenzone in the zeolite nanoparticle, significant scattering is observed, along with a broadening and bathochromic shift in absorption.

FIG. 4A is a plot of absorption spectra of a thin film of 1% AB dispersed in petroleum jelly on quartz plates over the course of 4 hours of irradiation using a UV photolysis lamp. A UV photolysis lamp having a light flux of 320 mW equipped with a cutoff filter at 270-280 nm was used to evaluate the performance and stability of the sunscreen compositions prepared herein. With 3 min of exposure to the UV lamp (same position as sample, after the filter), a clear damage was observed on human skin. Typically, under hot sun, this sunburn process takes 30 min to an hour. Accordingly, the photolysis lamp used for analysis is considerably more intense than natural sunlight.

FIG. 4B is a plot of absorption spectra of a thin film of avobenzone encapsulated in zeolite nanoparticles (10% zeolite, 1% AB) dispersed in petroleum jelly on quartz plates over the course of 4 hours of irradiation using a UV photolysis lamp.

FIG. 5 is a plot of absorption spectra of a thin film of avobenzone encapsulated in zeolite nanoparticles (10% zeolite, 1% AB) dispersed in petroleum jelly on quartz plates over the course of 4 hours of irradiation using a UV photolysis lamp. The surface of the zeolite nanoparticles was covalently modified with hexadecylamine (HDA) to render the sunscreen agent hydrophobic so that it disperses better in petroleum jelly. The contact angle of HDA-modified zeolite was 72.5°.

FIGS. 6A and 6B plot absorption spectra of a thin film of avobenzone encapsulated in zeolite nanoparticles (10% zeolite, 1% AB) dispersed in petroleum jelly on quartz plates over the course of 4 hours of irradiation using a UV photolysis lamp following storage in a desiccator for 15 days (FIG. 6A) and 28 days (FIG. 6B). The surface of the zeolite nanoparticles was covalently modified with hexadecylamine (HDA) to render the sunscreen agent hydrophobic so that it disperses better in petroleum jelly. The contact angle of HDA-modified zeolite was 72.5°

FIG. 7 is a plot showing the long-term stability of avobenzone/nanozeolite formulations. The absorption spectra of a thin film of avobenzone encapsulated in zeolite nanoparticles (10% zeolite, 1% AB) dispersed in petroleum jelly on quartz plates was obtained following 0, 3, 5, and 12 hours of irradiation using a UV photolysis lamp. As shown in FIG. 7, little to no degradation of the avobenzone was observed via UV spectroscopy.

FIGS. 8A and 8B show the spectra in the photolysis process for 4 hours of 1% OMC (octinoxate) and 1% AB in petroleum jelly (FIG. 8A) and 1% OMC and (10% zeolite, 1% AB) HDA-ABNZ in petroleum jelly (FIG. 8B). With both AB and OMC as free molecules in petroleum jelly, AB decomposition was observed after the first hour of photolysis. This decomposition was the result of the reaction between AB and OMC, as well as AB decompasition. By encapsulating AB in zeolite, AB and OMC are not accessible for reaction with each other, and the decomposition process is inhibited.

FIG. 9 is a drawing illustrating the encapsulation of DEET within the pores of zeolite nanoparticles.

FIG. 10 is a schematic illustration of the covalent modification of the surface of zeolite nanoparticles with 1,1,3,3-tetramethyldisilazane (TMDS). The surface modification of the zeolite nanoparticles can significantly impact the release rate of small molecules from the zeolite pores via ion-exchange. Notably, the release rate was found to vary based on the ionic strength of the solution surrounding the zeolite nanoparticles.

### DETAILED DESCRIPTION

Topical Compositions

Provided herein are topical compositions for the administration of active agents. The compositions can comprise zeolite nanoparticles dispersed in a topically acceptable carrier. The zeolite nanoparticles can further comprise an effective amount of an active agent adsorbed on the zeolite nanoparticles, encapsulated within the zeolite nanoparticles, or a combination thereof.

Zeolite Nanoparticles

The zeolite nanoparticles are generally aluminosilicate having a three-dimensionally grown skeleton structure and is generally shown by xM_{2/n}O.Al₂O₃.ySiO₂.zH₂O, wherein M represents an ion-exchangeable metal ion; n corresponds to the valence of the metal; x is a coefficient of the metal oxide; y is a coefficient of silica; and z is the number of water of crystallization. The zeolite nanoparticles can have varying frameworks and differing Si/Al ratios. In some embodiments, the zeolite nanoparticles can comprise zeolite having a faujasite structure. For example, the zeolite nanoparticles can be zeolite X or Y.

The zeolite nanoparticles can have an average particle size of less than 250 nm (e.g., less than 200 nm, less than 150 nm, less than 100 nm, less than 90 nm, less than 80 nm, less than 70 nm, less than 60 nm, less than 50 nm, less than 40 nm, less than 30 nm, or less than 20 nm). In some embodiments, the zeolite nanoparticles can have an average particle size of at least 10 nm (e.g., at least 20 nm, at least 30 nm, at least 40 nm, at least 50 nm, at least 60 nm, at least 70 nm, at least 80 nm, at least 90 nm, at least 100 nm, at least 150 nm, or at least 200 nm).

The zeolite nanoparticles can have an average particle size ranging from any of the minimum values described above to any of the maximum values described above. For example, in some embodiments, the zeolite nanoparticles can have an average particle size of from 10 to 250 nm (e.g., from 10 to 200 nm, from 10 to 150 nm, from 10 to 100 nm, from 20 to 80 nm, or from 20 to 60 nm).

The zeolite nanoparticles can possess a very regular pore structure of molecular dimensions. In some cases, the zeolite nanoparticles can exhibit a monodisperse pore size distribution. As used herein, a monodisperse pore size distribution refers to pore size distributions in which 80% of the distribution (e.g., 85% of the distribution, 90% of the distribution, or 95% of the distribution) lies within 20% of the median pore size (e.g., within 15% of the median pore size, within 10% of the median pore size, or within 5% of the median pore size).

In certain embodiments, the zeolite nanoparticles can exhibit an external pore size of 75 angstroms or less (e.g., 70 angstroms or less, 65 angstroms or less, 60 angstroms or less, 55 angstroms or less, 50 angstroms or less, 45 angstroms or less, 40 angstroms or less, 35 angstroms or less, 30 angstroms or less, 25 angstroms or less, 20 angstroms or less, or 15 angstroms or less). In certain embodiments, the zeolite nanoparticles can exhibit an external pore size of at least 10 angstroms (e.g., at least 15 angstroms, at least 20 angstroms, at least 25 angstroms, at least 30 angstroms, at least 35 angstroms, at least 40 angstroms, at least 45 angstroms, at least 50 angstroms, at least 55 angstroms, at least 60 angstroms, at least 65 angstroms, or at least 70 angstroms).

The zeolite nanoparticles can exhibit an external pore size of from any of the minimum values described above to any of the maximum values described above. For example, in some embodiments, the zeolite nanoparticles can exhibit an external pore size of from 10 to 75 angstroms (e.g., from 10 to 50 angstroms). In certain embodiments, the zeolite nanoparticles can exhibit an internal pore size of 8 angstroms or less (e.g., an internal pore size of from 2 to 8 angstroms).

The zeolite nanoparticles can also possess a high internal surface area. For example, in some embodiments, the zeolite nanoparticles can exhibit an average internal surface area of from 100 to 1,000 m²/g (e.g., from 200 to 1,000 m²/g, from 100 to 800 m²/g, from 200 to 800 m²/g, from 300 to 800 m²/g, from 300 to 700 m²/g, from 100 to 500 m²/g, from 200 to 500 m²/g, or from 400 to 800 m²/g).

In some cases, the zeolite nanoparticles can be modified, for example, to alter the surface chemistry of the zeolite nanoparticles. Depending on the active agent, zeolite, and the intended use of the composition, modification of the zeolite nanoparticles can alter the release characteristics of the active agent (e.g., stimulate release of the active agent, ensure encapsulation of the active agent), improve the dispersability of the zeolite nanoparticles in the topically acceptable carrier, increase the affinity of the zeolite nanoparticles for a target, or a combination thereof.

In some cases, the zeolite nanoparticles can have a hydrophobic surface (e.g., a surface that is covalently modified to increase its hydrophobicity). In some cases, the zeolite nanoparticles can have a hydrophilic surface (e.g., a surface that is covalently modified to increase its hydrophilicity). In some cases, the zeolite nanoparticles can have a charged surface (e.g., a surface that is modified to increase the zeta potential of the zeolite nanoparticles). In other cases, the zeolite nanoparticles can have a neutral surface.

Active Agents

The active agent can be any active agent that can be topically administered to a subject. The active agent can be, for example, a UV-blocking agent, antimicrobial agent, insecticide, cosmetic agent, fragrance, anesthetic agent, keratolytic agent, steroid, anthelmintic agent, dermatological agent, antioxidant, anti-inflammatory agent, or combination thereof.

In some case, the active agent can comprise metal nanoparticles. In some case, the active agent can comprise metal ions. In some case, the active agent can comprise a small molecule (e.g., an organic small molecule). “Small Molecule”, as used herein, refers to a molecule, such as an organic compound, with a molecular weight of less than about 2,000 Daltons (e.g., less than about 1,500 Daltons, less than about 1,000 Daltons, or less than about 800 Daltons).

In some embodiments, the active agent can have a molecular size of 13 Angstroms or less (e.g., 12 Angstroms or less, 11 Angstroms or less, 10 Angstroms or less, 9 Angstroms or less, 8 Angstroms or less, or 7 Angstroms or less). In certain embodiments, the active agent can have a molecular size of from 5 Angstroms to 13 Angstroms.

In some embodiments, the active agent can comprise a hydrophilic small molecule. In other embodiments, the active agent can comprise a hydrophobic small molecule. In some embodiments, the active agent can comprise a charged small molecule. In other embodiments, the active agent can comprise a neutral small molecule.

In certain embodiments, the active agent can comprise an insecticide (e.g., N,N-diethyl-meta-toluamide (DEET)). In certain embodiments, the active agent can be a UV-blocking agent (e.g., avobenzone, oxybenzone, or a combination thereof). In certain embodiments, the active agent can be an antimicrobial agent (e.g., silver nanoparticles, silver ions, copper ions, zinc ions, or a combination thereof).

The active agent can be present in an amount of at least 1% by weight (e.g., at least 5% by weight, at least 10% by weight, at least 15% by weight, at least 20% by weight, or at least 25% by weight), based on the total weight of the zeolite nanoparticles. In certain embodiments, the active agent can be present in an amount of 25% by weight or less (e.g., 22% by weight or less, 20% by weight or less, 15% by weight or less, 10% by weight or less, or 5% by weight or less), based on the total weight of the zeolite nanoparticles.

The active agent can be present in an amount ranging from any of the minimum values described above to any of the maximum values described above. For example, in some embodiments, the active agent can be present in an amount from 1% to 25% by weight (e.g., from 5% to 20% by weight, from 5% to 25% by weight, from 10% to 20% by weight, or from 15% to 25% by weight), based on the total weight of the zeolite nanoparticles.

In some embodiments, the active agent can be encapsulated within the zeolite nanoparticles. In certain embodiments, the active agent can be encapsulated within the zeolite nanoparticles, and the active agent remains encapsulated within the zeolite nanoparticles upon application of the composition to a subject's skin. In some embodiment, encapsulation of the active agent can stabilize the active agent against degradation (e.g., chemical degradation resulting from exposure to water, heat, sunlight, or a combination thereof) during storage and/or following application of the composition to a subject's skin. For example, in some cases, the active agent can be stable towards degradation for a period of at least 8 hours (e.g., at least 12 hours, or at least 24 hours, such as from 8 hours to 48 hours) upon application of the composition to the subject's skin. In another example, the active agent can be stable towards degradation for a period of at least two weeks (e.g., at least one month, at least six months, or at least one year) when stored at room temperature in the absence of light. In some embodiments, encapsulation of the active agent can sequester the active agent from the subject, thereby minimizing and/or eliminating the subject's allergic response to the active agent. In some embodiments, encapsulation of the active agent can sequester the active agent from other components of the composition, thereby allowing, for example, two active agents that undesirably react with one another (e.g., avobenzone and octinoxate) to be included in the same composition.

In some embodiments, the active agent is adsorbed on the zeolite nanoparticles, encapsulated within the zeolite nanoparticles, or a combination thereof, and the active agent can be released from the zeolite nanoparticles upon application of the composition to a subject's skin. In some embodiments, the zeolite nanoparticle can provide for the extended release of the active agent upon application of the composition to a subject's skin. For example, in some cases, the active agent can be released over an extended period of time (e.g., over a period of at least 4 hours, over a period of at least 8 hours, over a period of at least 24 hours) following application of the composition to a subject's skin.

Sunscreen Compositions

Prolonged exposure to ultraviolet (UV) radiation, such as from the sun, can lead to the formation of light dermatoses and erythema, as well as increase the risk of skin cancers, such as melanoma, and accelerate skin aging, such as loss of skin elasticity and wrinkling. Numerous sunscreen compositions are commercially available with varying ability to shield the body from ultraviolet light. However, numerous challenges still exist to provide sunscreen compositions that provide strong UV radiation protection.

To address these and other needs, provided herein are sunscreen agents, as well as compositions comprising these sunscreen agents dispersed in a topically acceptable carrier. The sunscreen agents can comprise an organic UV-blocking agent encapsulated within a porous inorganic nanomaterial.

In some embodiments, the sunscreen agent can comprise at least 1% by weight (e.g., at least 5% by weight, at least 10% by weight, at least 15% by weight, at least 20% by weight, or at least 25% by weight) UV-blocking agent, based on the total weight of the sunscreen agent. In some embodiments, the sunscreen agent can comprise 30% by weight or less (e.g., 25% by weight or less, 20% by weight or less, 15% by weight or less, 10% by weight or less, or 5% by weight or less) UV-blocking agent, based on the total weight of the sunscreen agent.

The sunscreen agent can comprise an amount of UV-blocking agent ranging from any of the minimum values described above to any of the maximum values described above. For example, in some embodiments, the sunscreen agent can comprise from 1% to 30% by weight (e.g., 5% to 20% by weight, or from 10% to 20% by weight) UV-blocking agent, based on the total weight of the sunscreen agent.

UV-Blocking Agents

The UV-blocking agent can be an organic compound that absorbs light in the UV region at one or more wavelengths from 290 nanometers (nm) to 400 nm. For example, the UV-blocking agent can exhibit a molar extinction coefficient of at least 10,000 mol⁻¹ L cm⁻¹ (e.g., at least 25,000 mol⁻¹ L cm⁻¹, at least 50,000 mol⁻¹ L cm⁻¹, at least 75,000 mol⁻¹ L cm⁻¹, or at least 100,000 mol⁻¹ L cm⁻¹) for at least one wavelength within the range of from 290 nm to 400 nm.

It is well documented that human skin is sensitive to sunlight and artificial light containing radiation of wavelengths between about 290 nanometers (nm) and 400 nm. Ultraviolet radiation of wavelengths between about 290 nm and 320 nm (UV-B region) has been known to rapidly produce damaging effects on the skin including reddening or erythema, edema, blistering or other skin eruptions in more severe cases. Prolonged or chronic exposure to radiation in this wavelength range has been associated with serious skin conditions such as actinic keratoses and carcinomas. In recent years, concern has also been expressed regarding ultraviolet radiation of wavelengths above 320 nm (UV-A region) and the adverse effects of such radiation on human skin. This damage potential is also the single most important cause of the premature aging of the skin. In addition, recent studies indicate that chronic sun exposure limits the immuno-response of the human body. There is also evidence that a tan will offer some protection against burning but is quite ineffectual against other types of solar damage.

In some embodiments, the UV-blocking agent can be an organic compound that absorbs light in the UV-B region at one or more wavelengths from 290 nm to 320 nm (i.e., a UV-B blocking agent). For example, the UV-blocking agent can exhibit a molar extinction coefficient of at least 10,000 mol⁻¹ L cm⁻¹ (e.g., at least 25,000 mol⁻¹ L cm⁻¹, at least 50,000 mol⁻¹ L cm⁻¹, at least 75,000 mol⁻¹ L cm⁻¹, or at least 100,000 mol⁻¹ L cm⁻¹) for at least one wavelength within the range of from 290 nm to 320 nm. In some cases, the UV-blocking agent can exhibit a molar extinction coefficient of at least 10,000 mol⁻¹ L cm⁻¹ at all wavelengths within the range of from 290 nm to 320 nm.

In some embodiments, the UV-blocking agent can be an organic compound that absorbs light in the UV-A region at one or more wavelengths from 320 nm to 400 nm (i.e., a UV-A blocking agent). For example, the UV-blocking agent can exhibit a molar extinction coefficient of at least 10,000 mol⁻¹ L cm⁻¹ (e.g., at least 25,000 mol⁻¹ L cm⁻¹, at least 50,000 mol⁻¹ L cm⁻¹, at least 75,000 mol⁻¹ L cm⁻¹, or at least 100,000 mol⁻¹ L cm⁻¹) for at least one wavelength within the range of from 320 nm to 400 nm. In some cases, the UV-blocking agent can exhibit a molar extinction coefficient of at least 10,000 mol⁻¹ L cm⁻¹ at all wavelengths within the range of from 320 nm to 400 nm.

Examples of suitable UV-blocking agents include, for example, p-aminobenzoic acid, padiate O, phenylbenzimidazole sulfonic acid, cinoxate, dixoybenzone, oxybenzone, homosalate, menthyl anthranilate, octocrylene, octyl methoxycinnamate, octyl salicylate, sulisobenzone, trolamine salicylate, avobenzone, ecamsule, 4-methylbenzylidene camphor, bisoctrizole, bemotrizinol, bisdisulizole disodium, tris-biphenyl triazine, drometrizole trisiloxane, benzophenone-9, ethylhexyl triazone, diethylamino hydroxybenzoyl hexyl benzoate, iscotrizinol, polysilicone-15, amiloxate, and combinations thereof. In some embodiments, the UV-blocking agent can be p-aminobenzoic acid, padiate O, phenylbenzimidazole sulfonic acid, cinoxate, dixoybenzone, oxybenzone, homosalate, menthyl anthranilate, octocrylene, octyl methoxycinnamate, octyl salicylate, sulisobenzone, trolamine salicylate, avobenzone, ecamsule, or a combination thereof. In certain embodiments, the UV-blocking agent can be avobenzone, oxybenzone, or a combination thereof.

Nanomaterials

The porous inorganic nanomaterial can be nanoparticles formed from a microporous or mesoporous inorganic material. Preferably, the porous inorganic nanomaterial can be capable of scattering UV light.

In some embodiments, the porous inorganic nanomaterial can have an average particle size of less than 250 nm (e.g., less than 200 nm, less than 150 nm, less than 100 nm, less than 90 nm, less than 80 nm, less than 70 nm, less than 60 nm, less than 50 nm, less than 40 nm, less than 30 nm, or less than 20 nm). In some embodiments, the porous inorganic nanomaterial can have an average particle size of at least 10 nm (e.g., at least 20 nm, at least 30 nm, at least 40 nm, at least 50 nm, at least 60 nm, at least 70 nm, at least 80 nm, at least 90 nm, at least 100 nm, at least 150 nm, or at least 200 nm).

The porous inorganic nanomaterial can have an average particle size ranging from any of the minimum values described above to any of the maximum values described above. For example, in some embodiments, the porous inorganic nanomaterial can have an average particle size of from 10 to 250 nm (e.g., from 10 to 100 nm, or from 10 to 50 nm).

The porous inorganic nanomaterial can possesses a very regular pore structure of molecular dimensions. In some cases, the porous inorganic nanomaterial can exhibit a monodisperse pore size distribution. As used herein, a monodisperse pore size distribution refers to pore size distributions in which 80% of the distribution (e.g., 85% of the distribution, 90% of the distribution, or 95% of the distribution) lies within 20% of the median pore size (e.g., within 15% of the median pore size, within 10% of the median pore size, or within 5% of the median pore size).

In certain embodiments, the porous inorganic nanomaterial can exhibit a pore size of 75 angstroms or less (e.g., 70 angstroms or less, 65 angstroms or less, 60 angstroms or less, 55 angstroms or less, 50 angstroms or less, 45 angstroms or less, 40 angstroms or less, 35 angstroms or less, 30 angstroms or less, 25 angstroms or less, 20 angstroms or less, or 15 angstroms or less). In certain embodiments, the porous inorganic nanomaterial can exhibit a pore size of at least 10 angstroms (e.g., at least 15 angstroms, at least 20 angstroms, at least 25 angstroms, at least 30 angstroms, at least 35 angstroms, at least 40 angstroms, at least 45 angstroms, at least 50 angstroms, at least 55 angstroms, at least 60 angstroms, at least 65 angstroms, or at least 70 angstroms).

The porous inorganic nanomaterial can exhibit a pore size of from any of the minimum values described above to any of the maximum values described above. For example, in some embodiments, the porous inorganic nanomaterial can exhibit a pore size of from 10 to 75 angstroms (e.g., from 10 to 50 angstroms).

The porous inorganic nanomaterial can also possess a high internal surface area. For example, in some embodiments, the porous inorganic nanomaterial can exhibit an internal surface area of from 100 to 1,000 m²/g (e.g., from 200 to 1,000 m²/g, from 100 to 800 m²/g, from 200 to 800 m²/g, from 100 to 500 m²/g, from 200 to 500 m²/g, or from 500 to 1,000 m²/g,).

In some embodiments, the porous inorganic nanomaterial can comprise alumino-silicate nanoparticles (e.g., zeolite nanoparticles). In certain embodiments, the porous inorganic nanomaterial can comprise zeolite nanoparticles having a faujasite structure. In other embodiments, the porous inorganic nanomaterial comprises nanoparticles formed from a metal-organic framework. The metal-organic framework can be, for example, an iron(III) dicarboxylate framework, an iron(III) tetramethylterephthalate framework, an iron(III) muconate framework, a zinc terephthalate framework, a zinc imidazolate framework, or a combination thereof. Suitable metal organic frameworks are known in the art, and include, for example, metal-organic frameworks such as MIL-88A, MIL-88B-4CH3, MIL-89, MIL-100(Fe), MIL-53(Fe), MOF-5, ZIF-8, and combinations thereof.

In some embodiments, the porous inorganic nanomaterial can be hydrophobically modified, meaning that the nanomaterial can be modified to increase the hydrophobicity of a surface of the nanomaterial. By increasing the hydrophobicity of the porous inorganic nanomaterial, the dispersability of the porous inorganic nanomaterial in hydrophobic carriers can be increased. In addition, compositions (e.g., sunscreens) containing hydrophobically modified materials can be more water/sweat resistant once applied.

The porous inorganic nanomaterial can be hydrophobically modified in any suitable fashion. In certain cases, the porous inorganic nanomaterial can be covalently modified to increase its hydrophobicity. Appropriate methods for modifying a porous inorganic nanomaterial to increase its hydrophobicity can be selected based on the chemical characteristics of the porous inorganic nanomaterial.

By way of example, in some cases, the porous inorganic nanomaterial can comprise alumino-silicate nanoparticles (e.g., zeolite nanoparticles) whose surfaces are covalently modified to increase their hydrophobicity. Alumino-silicate nanoparticles (e.g., zeolite nanoparticles) can be covalently modified by, for example, reacting the nanoparticles with a suitable hydrophobic silane. Suitable silane reagents that can be used to covalently modify alumino-silicate nanoparticles include silanes that contain a hydrolysable functional group (chloro-, alkoxy-, etc.).

Examples of suitable alkoxysilanes that can be used to covalently modify alumino-silicate nanoparticles (e.g., zeolite nanoparticles) include methyl triethoxysilane, methyl trimethoxysilane, methyl triphenoxysilane, propyl triphenoxysilane, methyl tricyclopentoxysilane, propyl tricyclohexoxy silane, methyl tricyclooctoxysilane, propyl diethoxy phenoxysilane, methyl tripropoxysilane, methyl tri-n-amyloxysilane, propyl triisopropoxysilane, ethyl triethoxysilane, diethyl diethoxysilane, isopropyl triethoxysilane, n-butyl triethoxysilane, n-amyl triethoxysilane, n-amyl trimethoxysilane, phenyl triethoxysilane, cyclopentyl triethoxysilane, cyclohexyl triethoxysilane, cyclooctyl triethoxysilane, dimethyl diethoxysilane, methyl ethyl diethoxysilane, tri(n-propyl)ethoxysilane, n-propyl trimethoxysilane, n-propyl triethoxysilane, di(n-propyl)diethoxysilane, trimethyl ethoxysilane, diphenyl diethoxysilane, diethyl diethoxysilane, n-octyl triethoxysilane, methyl tri(methoxyethoxy)silane, propyl tri(ethoxyethoxy)silane, 1H,1H,2H,2H-perfluorooctyltriethoxysilane, trimethoxy(octadecyl)silane, triethoxy(octyl)silane, and trialkoxycaprylylsilanes (e.g., trimethoxycaprylylsilane). Examples of suitable chlorosilanes that can be used to covalently modify alumino-silicate nanoparticles (e.g., zeolite nanoparticles) include octadecyltrichlorosilane (OTS), octadecyltrichlorosilane (OTS), hexyltrichlorosilane (HTS), and ethyltrichlorosilane (ETS).

In some embodiments, the porous inorganic nanomaterial can comprise an alumino-silicate nanoparticle (e.g., a zeolite nanoparticle) whose surface has been covalently modified with a caprylylsilane (e.g., with a trialkoxycaprylylsilane such as trimethoxycaprylylsilane) to increase its hydrophobicity.

In some embodiments, the porous inorganic nanomaterial can further comprise a quenching species. The quenching species can comprise a quenching ion. For example, the quenching ion can be an ion introduced by ion exchange into the porous inorganic nanomaterial (e.g., into the zeolite nanoparticle). Examples of suitable quenching ions include cations, such as alkali metal ions, transition metal ions, rare earth ions, and combinations thereof. The quenching species can also be an organic molecule, such as nitromethane, an amine compound, or a combination thereof.

Compositions

Also provided herein are compositions comprising the sunscreen agents described above dispersed in a topically acceptable carrier. As used herein, “topically acceptable” means suitable for use in contact with tissues (e.g., the skin) without undue toxicity, incompatibility, instability, irritation, allergic response, or the like. In one embodiment, a composition suitable for topical/cosmetic use for application to the human body (e.g., keratinaceous surfaces such as the skin, hair, lips, or nails), especially the skin, is provided. The composition can be appropriately formulated for topical application to a subject (e.g., for application to the skin of a subject). For example, the composition can be a cream, dispersion, emulsion, gel, ointment, lotion, milk, mousse, spray, or tonic. In some embodiments, the composition can be a sunscreen or cosmetic.

The term “minimal erythema dose” (MED) refers to the quantity of erythema-effective energy (expressed as Joules per square meter or milli joules per square centimeter) required to produce the first perceptible, redness reaction in the skin with clearly defined borders.

Guidelines for labelling of Sun Protection Factor (SPF) and product categories are suggested by COLIPA (The European Cosmetic Toiletry and perfumery Association) in Europe. These are as listed in the table below:

| Labeled Category | Labeled SPF | Measured SPF |
|---|---|---|
| Low Protection | 6 | 6.0-9.9 |
| | 10 | 10.0-14.9 |
| Medium Protection | 15 | 15.0-19.9 |
| | 20 | 20.0-24.9 |
| | 25 | 25.0-29.9 |
| High Protection | 30 | 30.0-49.9 |
| | 50 | 50.0-59.9 |
| Very high protection | 50+ | ≥60 |

The term “Sun protection factor” (SPF) refers to the UV energy required to produce an MED on protected skin divided by the UV energy required to produce an MED on unprotected skin. The “sun protection factor” term may also be defined as the ratio of the minimum erythemal dose on protected skin (MEDp) to the minimum erythemal dose on unprotected skin (MED_{U}):

SPF=MED_{P}/MEDᵤ

The Sun Protection Factor value on an individual subject (SPFi), for any product or composition, either before or after water immersion, may be determined as the ratio of the minimum erythemal dose on protected skin (MED_{P}) to the minimum erythemal dose on unprotected skin (MED_{U}) of the same subject.

Further, the term “static sun protection factor”, (SPF_{S}), relates to the sun protection factor before water immersion, while the term “wet sun protection factor” (SPFw) refers to the sun protection factor after water immersion.

The static and wet SPF values are determined according to the current published International Sun Protection Factor (SPF) Test Method (I-SPF-TM) as defined in 2006 by COLIPA (CTFA SA-JCIA-CFTA US) as well as international standard ISO 24444: 2010(E). In some embodiments, the compositions can be formulation to exhibit an SPF of at least 15 (e.g., at least 30), as measured using the international standard ISO 24444: 2010(E).

Compositions can include one or more sunscreen agents described herein. The concentration of the sunscreen agents may vary from 0.5% to 50% by weight (e.g., from 5% to 40% by weight, from 10% to 25% by weight, from 0.5% to 30% by weight, from 0.5% to 20% by weight or from 0.5% to 10% by weight) of the composition, based on the total weight of the composition.

The compositions described herein be used for a variety of cosmetic uses, especially for protection of the skin from UV radiation. The compositions, thus, may be made into a wide variety of delivery forms. These forms include, but are not limited to, suspensions, dispersions, solutions, or coatings on water soluble or water-insoluble substrates (e.g., substrates such as organic or inorganic powders, fibers, or films). The composition may be employed for various end-uses, such as recreation or daily-use sunscreens, moisturizers, cosmetics/make-up, cleansers/toners, anti-aging products, or combinations thereof. These compositions may be prepared using methodologies that are known in the field of cosmetics formulation.

Antimicrobial Compositions

Also provided herein are antimicrobial agents, compositions comprising these antimicrobial agents, as well as methods of making and using them. As used herein, “antimicrobial” refers to the ability to treat or control (e.g., reduce, prevent, treat, or kill) the growth of a microbe at any concentration. The microbe may be a bacteria, a fungi, an algae, a virus, or a combination thereof. Thus, the term antimicrobial encompasses “antibacterial,” “antifungal,” and “antiviral,” which refer to the ability to treat or control the growth of bacteria, fungi, and viruses at any concentration, respectively. The antimicrobial agents described herein comprise zeolite nanoparticles, wherein the zeolite nanoparticles further comprise silver.

Zeolite Nanoparticles

The zeolite nanoparticles are generally aluminosilicate having a three-dimensionally grown skeleton structure and is generally shown by xM_{2/n}O.Al₂O₃.ySiO₂.zH₂O, wherein M represents an ion-exchangeable metal ion; n corresponds to the valence of the metal; x is a coefficient of the metal oxide; y is a coefficient of silica; and z is the number of water of crystallization. The zeolite nanoparticles can have varying frameworks and differing Si/Al ratios. In some embodiments, the zeolite nanoparticles can comprise zeolite having a faujasite structure. For example, the zeolite nanoparticles can be zeolite X or Y.

The zeolite nanoparticles can have an average particle size of less than 250 nm (e.g., less than 200 nm, less than 150 nm, less than 100 nm, less than 90 nm, less than 80 nm, less than 70 nm, less than 60 nm, less than 50 nm, less than 40 nm, less than 30 nm, or less than 20 nm). In some embodiments, the zeolite nanoparticles can have an average particle size of at least 10 nm (e.g., at least 20 nm, at least 30 nm, at least 40 nm, at least 50 nm, at least 60 nm, at least 70 nm, at least 80 nm, at least 90 nm, at least 100 nm, at least 150 nm, or at least 200 nm).

The zeolite nanoparticles can have an average particle size ranging from any of the minimum values described above to any of the maximum values described above. For example, in some embodiments, the zeolite nanoparticles can have an average particle size of from 10 to 250 nm (e.g., from 10 to 200 nm, from 10 to 150 nm, from 10 to 100 nm, from 20 to 80 nm, or from 20 to 60 nm).

The zeolite nanoparticles can possess a very regular pore structure of molecular dimensions. In some cases, the zeolite nanoparticles can exhibit a monodisperse pore size distribution. As used herein, a monodisperse pore size distribution refers to pore size distributions in which 80% of the distribution (e.g., 85% of the distribution, 90% of the distribution, or 95% of the distribution) lies within 20% of the median pore size (e.g., within 15% of the median pore size, within 10% of the median pore size, or within 5% of the median pore size).

In certain embodiments, the zeolite nanoparticles can exhibit an external pore size of 75 angstroms or less (e.g., 70 angstroms or less, 65 angstroms or less, 60 angstroms or less, 55 angstroms or less, 50 angstroms or less, 45 angstroms or less, 40 angstroms or less, 35 angstroms or less, 30 angstroms or less, 25 angstroms or less, 20 angstroms or less, or 15 angstroms or less). In certain embodiments, the zeolite nanoparticles can exhibit an external pore size of at least 10 angstroms (e.g., at least 15 angstroms, at least 20 angstroms, at least 25 angstroms, at least 30 angstroms, at least 35 angstroms, at least 40 angstroms, at least 45 angstroms, at least 50 angstroms, at least 55 angstroms, at least 60 angstroms, at least 65 angstroms, or at least 70 angstroms).

The zeolite nanoparticles can exhibit an external pore size of from any of the minimum values described above to any of the maximum values described above. For example, in some embodiments, the zeolite nanoparticles can exhibit an external pore size of from 10 to 75 angstroms (e.g., from 10 to 50 angstroms). In certain embodiments, the zeolite nanoparticles can exhibit an internal pore size of 8 angstroms or less (e.g., an internal pore size of from 2 to 8 angstroms).

The zeolite nanoparticles can also possess a high internal surface area. For example, in some embodiments, the zeolite nanoparticles can exhibit an average internal surface area of from 100 to 1,000 m²/g (e.g., from 200 to 1,000 m²/g, from 100 to 800 m²/g, from 200 to 800 m²/g, from 300 to 800 m²/g, from 300 to 700 m²/g, from 100 to 500 m²/g, from 200 to 500 m²/g, or from 400 to 800 m²/g).

The ion-exchange capacities of the zeolite nanoparticles may depend on the silica/aluminum ratio in their formulation. Zeolite types with low silica/aluminum ratios generally exhibit high ion-exchange capacities. In some embodiments, the SiO₂/Al₂O₃ mole ratio in the zeolite nanoparticles is 14 or less (e.g., 13 or less, 12 or less, 11 or less, 10 or less, 9 or less, 8 or less, 7 or less, 6 or less, or 5 or less). In some embodiments, the zeolite nanoparticles can retain a metal ion in an amount as large as or less than an ion-exchange saturation capacity of the zeolite nanoparticles.

Silver

As described herein, the zeolite nanoparticles comprise silver. The silver can kill or inhibit the growth of a microbe. In some embodiments, the silver present in the zeolite nanoparticles can comprise silver nanoparticles. The silver nanoparticles are suitable as silver metal nanoparticles that have antimicrobial activity.

The silver nanoparticles can have an average particle size of 15 nm or less (e.g., 10 nm or less, 9 nm or less, 8 nm or less, 7 nm or less, 6 nm or less, 5 nm or less, 4 nm or less, 3 nm or less, 2 nm or less, or even 1 nm). In certain embodiments, the silver nanoparticles can have an average particle size of at least 1 nm (e.g., at least 2 nm, at least 3 nm, at least 4 nm, at least 5 nm, at least 6 nm, at least 7 nm, at least 8 nm, at least 9 nm, or up to 10 nm).

The silver nanoparticles can have an average particle size ranging from any of the minimum values described above to any of the maximum values described above. For example, in some embodiments, the silver nanoparticles can have an average particle size of from 1 to 10 nm (e.g., from 1 to 8 nm, or from 1 to 5 nm).

The silver nanoparticles can be present in an amount of at least 1% by weight (e.g., at least 5% by weight, at least 10% by weight, at least 15% by weight, at least 20% by weight, or at least 25% by weight), based on the total weight of the zeolite nanoparticles and silver. In certain embodiments, the silver nanoparticles can be present in an amount of 25% by weight or less (e.g., 22% by weight or less, 20% by weight or less, 15% by weight or less, 10% by weight or less, or 5% by weight or less), based on the total weight of the zeolite nanoparticles and silver.

The silver nanoparticles can be present in an amount ranging from any of the minimum values described above to any of the maximum values described above. For example, in some embodiments, the silver nanoparticles can be present in an amount from 1% to 25% by weight (e.g., from 5% to 20% by weight, from 5% to 25% by weight, from 10% to 20% by weight, or from 15% to 25% by weight), based on the total weight of the zeolite nanoparticles and silver.

In some cases, the silver in the antimicrobial compositions can comprise silver ions. The silver ions can be retained at the ion exchangeable sites of the zeolite nanoparticles. That is, the ion-exchangeable ions such as sodium ions, calcium ions, potassium ions, magnesium ions and/or iron ions in the zeolite nanoparticles can be partially or wholly replaced with the silver ions.

The silver ions can be present in an amount as large as or less than the ion-exchange saturation capacity of the zeolite nanoparticles. In some embodiments, the zeolite nanoparticles retain silver ions in an amount of 10% or greater, 15% or greater, 20% or greater, 25% or greater, 30% or greater, 40% or greater, 50% or greater, 60% or greater, 75% or greater, 80% or greater, 90% or greater, 95% or greater, or up to 100%, of the ion exchange capacity of the zeolite nanoparticles. In some embodiments, the zeolite nanoparticles can retain the silver ions in an amount of 100% or less, 95% or less, 90% or less, 85% or less, 75% or less, 50% or less, 40% or less, or 25% or less, of the ion exchange capacity of the zeolite nanoparticles.

The silver ions can be present in an amount ranging from any of the minimum values described above to any of the maximum values described above. For example, in some embodiments, the silver ions can be retained in an amount from 10% up to 100% by weight (e.g., from 20% up to 100%, from 30% up to 100%, from 40% up to 100%, or from 50% up to 100%), of the ion exchange capacity of the zeolite nanoparticles.

In some cases, the zeolite nanoparticles can include silver nanoparticles in addition to silver ions. The silver nanoparticles and the silver ions can be present in an amount ranging from any of the minimum values described above to any of the maximum values described above. For example, in some embodiments, the zeolite nanoparticles can include (a) silver nanoparticles present in an amount from 1% to 25% by weight (e.g., 5% to 20% by weight, or from 15% to 25% by weight), based on the total weight of the zeolite nanoparticles and silver and (b) silver ions retained in an amount of 100% or less, 95% or less, 90% or less, 85% or less, 75% or less, of the ion exchange capacity of the zeolite nanoparticles.

Adjuvants

The zeolite nanoparticles described herein can comprise, in addition to silver, an adjuvant. The term “adjuvant” as described herein refers to a substance added to or co-formulated with the compositions described herein to enhance, induce, elicit, and/or modulate the antimicrobial activity of silver when contacted to a microbe.

In some embodiments, the adjuvant comprises antimicrobial metal ions. The antimicrobial metal ions can include a metal selected from copper ions, zinc ions, mercury ions, lead ions, tin ions, bismuth ions, cadmium ions, chromium ions, antimony ions, arsenic ions, or thallium ions. In some examples, the adjuvant can include copper ions, zinc ions, or a combination thereof. The antimicrobial metal ions can be present in an amount of 100% or less, 95% or less, 90% or less, 85% or less, 75% or less, 70% or less, 60% or less, 50% or less, 40% or less, 30% or less, 25% or less, 20% or less, 15% or less, 10% or less, of the ion exchange capacity of the zeolite nanoparticles.

In some embodiments, the adjuvant comprises hydrogen ions. The hydrogen ions can be present in an amount to reduce the pH of an aqueous region in contact with the zeolite nanoparticles.

In some embodiments, the adjuvant comprises a small molecule antimicrobial agent. “Small Molecule”, as used herein, refers to a molecule, such as an organic compound, with a molecular weight of less than about 2,000 Daltons (e.g., less than about 1,500 Daltons, less than about 1,000 Daltons, or less than about 800 Daltons). The small molecule antimicrobial agent can be selected from an antibacterial agent, an antiviral agent, and/or an antifungal agent. Suitable examples of small molecule antimicrobial agent include antibiotics, disinfectant, antiseptics, or a combination thereof. In certain embodiments, the small molecule antimicrobial agent can include a hydrophilic small molecule.

Representative examples of small molecule antimicrobial agents include, for example, alexidine, asphodelin A, atromentin, auranthine, austrocortilutein, austrocortirubin, azerizin, chlorbisan, chloroxine, cidex, cinoxacin, citreorosein, copper usnate, cupiennin, curvularin, DBNPA, dehydrocurvularin, desoxyfructo-serotonin, dichloroisocyanuric acid, elaiomycin, holtfreter's solution, malettinin, naphthomycin, neutrolin, niphimycin, nitrocefin, oxadiazoles, paenibacterin, proclin, ritiometan, ritipenem, silicone quaternary amine, stylisin, taurolidine, tirandamycin, trichloroisocyanuric acid, and triclocarban.

Examples of antibacterials include, for example, acetoxycycloheximide, aciduliprofundum, actaplanin, actinorhodin, alazopeptin, albomycin, allicin, allistatin, allyl isothiocyanate, ambazone, aminocoumarin, aminoglycosides, 4-aminosalicylic acid, ampicillin, ansamycin, anthramycin, antimycin A, aphidicolin, aplasmomycin, archaeocin, arenicin, arsphenamine, arylomycin A2, ascofuranone, aspergillic acid, avenanthramide, avibactam, azelaic acid, bafilomycin, bambermycin, beauvericin, benzoyl peroxide, blasticidin S, bottromycin, brilacidin, caprazamycin, carbomycin, cathelicidin, cephalosporins, ceragenin, chartreusin, chromomycin A3, citromycin, clindamycin, clofazimine, clofoctol, clorobiocin, coprinol, coumermycin A1, cyclic lipopeptides, cycloheximide, cycloserine, dalfopristin, dapsone, daptomycin, debromomarinone, 17-dimethylaminoethylamino-17-demethoxygeldanamycin, echinomycin, endiandric acid C, enediyne, enviomycin, eravacycline, erythromycin, esperamicin, etamycin, ethambutol, ethionamide, (6S)-6-fluoroshikimic acid, fosfomycin, fosmidomycin, friulimicin, furazolidone, furonazide, fusidic acid, geldanamycin, gentamycin, gepotidacin, glycyciclines, glycyrrhizol, gramicidin S, guanacastepene A, hachimycin, halocyamine, hedamycin, helquinoline, herbimycin, hexamethylenetetramine, hitachimycin, hydramacin-1, isoniazid, kanamycin, katanosin, kedarcidin, kendomycin, kettapeptin, kidamycin, lactivicin, lactocillin, landomycin, landomycinone, lasalocid, lenapenem, leptomycin, lincosamides, linopristin, lipiarmycins, macbecin, macrolides, macromomycin B, maduropeptin, mannopeptimycin glycopeptide, marinone, meclocycline, melafix, methylenomycin A, methylenomycin B, monensin, moromycin, mupirocin, mycosubtilin, myriocin, myxopyronin, naphthomycin A, narasin, neocarzinostatin, neopluramycin, neosalvarsan, neothramycin, netropsin, nifuroxazide, nifurquinazol, nigericin, nitrofural, nitrofurantoin, nocathiacin I, novobiocin, omadacycline, oxacephem, oxazolidinones, penicillins, peptaibol, phytoalexin, plantazolicin, platensimycin, plectasin, pluramycin A, polymixins, polyoxins, pristinamycin, pristinamycin IA, promin, prothionamide, pulvinone, puromycin, pyocyanase, pyocyanin, pyrenocine, questiomycin A, quinolones, quinupristin, ramoplanin, raphanin, resistome, reuterin, rifalazil, rifamycins, ristocetin, roseophilin, salinomycin, salinosporamide A, saptomycin, saquayamycin, seraticin, sideromycin, sodium sulfacetamide, solasulfone, solithromycin, sparassol, spectinomycin, staurosporine, streptazolin, streptogramin, streptogramin B, streptolydigin, streptonigrin, styelin A, sulfonamides, surfactin, surotomycin, tachyplesin, taksta, tanespimycin, telavancin, tetracyclines, thioacetazone, thiocarlide, thiolutin, thiostrepton, tobramycin, trichostatin A, triclosan, trimethoprim, trimethoprim, tunicamycin, tyrocidine, urauchimycin, validamycin, viridicatumtoxin B, vulgamycin, xanthomycin A, and xibornol.

Examples of antifungals include, for example, abafungin, acibenzolar, acibenzolar-S-methyl, acrisorcin, allicin, aminocandin, amorolfine, amphotericin B, anidulafungin, azoxystrobin, bacillomycin, Bacillus pumilus, barium borate, benomyl, binapacryl, boric acid, bromine monochloride, bromochlorosalicylanilide, bupirimate, butenafine, candicidin, caprylic acid, captafol, captan, carbendazim, caspofungin, cerulenin, chloranil, chlormidazole, chlorophetanol, chlorothalonil, chloroxylenol, chromated copper arsenate, ciclopirox, cilofungin, cinnamaldehyde, clioquinol, copper(I) cyanide, copper(II) arsenate, cruentaren, cycloheximide, davicil, dehydroacetic acid, dicarboximide fungicides, dichlofluanid, dimazole, diphenylamine, echinocandin, echinocandin B, epoxiconazole, ethonam, falcarindiol, falcarinol, famoxadone, fenamidone, fenarimol, fenpropimorph, fentin acetate, fenticlor, filipin, fluazinam, fluopicolide, flusilazole, fluxapyroxad, fuberidazole, griseofulvin, halicylindramide, haloprogin, hamycin, hexachlorobenzene, hexachlorocyclohexa-2,5-dien-1-one, 5-hydroxy-2(5H)-furanone, iprodione, lime sulfur, mancozeb, maneb, melafix, metalaxyl, metam sodium, methylisothiazolone, methylparaben, micafungin, miltefosine, monosodium methyl arsenate, mycobacillin, myclobutanil, natamycin, beta-nitrostyrene, nystatin, paclobutrazol, papulacandin B, parietin, pecilocin, pencycuron, pentamidine, pentachloronitrobenzene, pentachlorophenol, perimycin, 2-phenylphenol, polyene antimycotic, propamocarb, propiconazole, pterulone, ptilomycalin A, pyrazophos, pyrimethanil, pyrrolnitrin, selenium disulfide, sparassol, strobilurin, sulbentine, tavaborole, tebuconazole, terbinafine, theonellamide F, thymol, tiabendazole, ticlatone, tolciclate, tolnaftate, triadimefon, triamiphos, tribromometacresol, 2,4,6-tribromophenol, tributyltin oxide, triclocarban, triclosan, tridemorph, trimetrexate, undecylenic acid, validamycin, venturicidin, vinclozolin, vinyldithiin, vusion, xanthene, zinc borate, zinc pyrithione, zineb and ziram.

Examples of antivirals include, for example, afovirsen, alisporivir, angustific acid, angustifodilactone, alovudine, beclabuvir, 2,3-bis(acetylmercaptomethyl)quinoxaline, brincidofovir, dasabuvir, docosanol, fialuridine, ibacitabine, imiquimod, inosine, inosine pranobex, interferon, metisazone, miltefosine, neokadsuranin, neotripterifordin, ombitasvir, oragen, oseltamivir, pegylated interferon, podophyllotoxin, radalbuvir, semapimod, tecovirimat, telbivudine, theaflavin, tilorone, triptofordin C-2, variecolol and ZMapp.

The small molecule antimicrobial agent can be present in an amount from 0.1% to 20% by weight (e.g., from 0.1% to 20% by weight, from 0.1% to 15% by weight, from 0.1% to 10% by weight, or from 0.1% to 5% by weight), based on the total weight of the zeolite nanoparticles and silver.

Targeting Agents

The zeolite nanoparticles can also include a microbial targeting agent. The microbial targeting agent can be covalently linked to the zeolite nanoparticles. Some microbes are known to have a negative charge density on their surface. Therefore, in some embodiments, the microbial targeting agent can comprise a cationic group or a cationic precursor. In some embodiments, the microbial targeting agent can comprise an amine containing group. The amine containing group can include an alkyl amine such as a C₁-C₁₂ alkyl amine.

The microbial targeting agent can be present in an amount from 0.1% to 20% by weight (e.g., from 1% to 20% by weight, from 1% to 15% by weight, from 1% to 10% by weight, or from 0.1% to 5% by weight), based on the total weight of the zeolite nanoparticles and silver.

In some embodiments of the antimicrobial agents disclosed herein, the antimicrobial agent can include zeolite nanoparticles, wherein the zeolite nanoparticles further comprise silver nanoparticles disposed on and/or within the zeolite nanoparticles. In other embodiments of the antimicrobial agents disclosed herein, the antimicrobial agent can include zeolite nanoparticles, wherein the zeolite nanoparticles further comprise silver nanoparticles disposed on and/or within the zeolite nanoparticles and antimicrobial metal ions retained at ion-exchangeable sites within the zeolite nanoparticles. In further embodiments of the antimicrobial agents disclosed herein, the antimicrobial agent can include zeolite nanoparticles, wherein the zeolite nanoparticles further comprise silver nanoparticles disposed on and/or within the zeolite nanoparticles and wherein a surface of the zeolite nanoparticles is functionalized with a microbial targeting agent. In still further embodiments of the antimicrobial agents disclosed herein, the antimicrobial agent can include zeolite nanoparticles, wherein the zeolite nanoparticles further comprise silver nanoparticles disposed on and/or within the zeolite nanoparticles and a small molecule antimicrobial agent adsorbed on and/or within the zeolite nanoparticle.

Compositions

Also provided herein are compositions comprising the antimicrobial agents described herein. Depending on the intended application or mode of administration, the antimicrobial compositions can be in the form of solid, semi-solid or liquid forms, such as, for example, powders, liquids, dispersion, or suspensions.

In some examples, the compositions can comprise the antimicrobial agents and a carrier. In some embodiments, the carrier can be a pharmaceutically acceptable carrier. As used herein, “pharmaceutically acceptable” refers to a material that is not biologically or otherwise undesirable, which can be administered to an individual along with the selected compositions without causing unacceptable biological effects or interacting in a deleterious manner with the other components of the compositions in which it is contained. In some examples, the compositions further comprising pharmaceutically acceptable carrier are referred to as pharmaceutically acceptable formulations. A pharmaceutically acceptable formulation refers to those formulations of the compositions described herein that are within the scope of sound medical judgment, suitable for use in contact with the tissues of subjects without undue toxicity, irritation, allergic response, and the like, commensurate with a reasonable benefit/risk ratio, and effective for their intended use.

As used herein, the term carrier encompasses any excipient, diluent, filler, salt, buffer, stabilizer, solubilizer, lipid, surfactant, solvent, thickener, wax, cement, plaster, adhesive, coating, or other material well known in the art for use in applications as described herein. The choice of a carrier for use in the composition will depend upon the intended application or route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN′ (ICI, Inc.; Bridgewater, N.J.), polyethylene glycol (PEG), and PLURONICS™ (BASF; Florham Park, N.J.).

The compositions can include the antimicrobial agents in an amount of from 0.1% to 99% by weight (e.g., from 5% to 90% by weight, from 5% to 80% by weight, or from 5% to 60% by weight), based on the total weight of the composition.

Methods of Making

Methods of making the antimicrobial agents are also disclosed. Methods of making zeolite nanoparticles are described in PCT/2015/060681, which is incorporated herein by reference in its entirety. Briefly, the method can include (a) heating a first mixture comprising a silicon source, an aluminum source, a base, an organic agent, and a first solvent to produce a first population of zeolite nanoparticles dispersed in a first supernatant; (b) separating the first population of zeolite nanoparticles from the first supernatant; (c) adding a base to the first supernatant to form a second mixture; (d) heating the second mixture to produce a second population of zeolite nanoparticles dispersed in a second supernatant; and (e) separating the second population of zeolite nanoparticles from the second supernatant. The first population of zeolite nanoparticles and the second population of zeolite nanoparticles prepared by the methods described herein can each have an average particle size of 250 nm or less, such as 100 nm or less.

In certain embodiments, the methods of making the zeolite nanoparticles can include mixing a silicon source, an aluminum source, a base, an organic agent, and a first solvent to form a first mixture. The silicon and/or aluminum source can include any suitable compound that will hydrolyze to provide silicon and/or aluminum to form the framework of the zeolite nanoparticles. For example, the silicon source can include tetraethylorthosilane (TEOS), colloidal or fumed silica (amorphous silica such as Ludox LS30), disodium metasilicate, or combinations thereof. The aluminum source can include aluminum hydroxide, aluminum isopropoxide, sodium aluminate, aluminum sulfate, or combinations thereof. The organic agent can be a porous material that can serve as the structure around which an alumino-silicate nanoparticles can form. For example, the organic agent can be any suitable organic base. Examples of organic agents can include tetrapropyl ammonium hydroxide (TPAOH), tetramethyl ammonium hydroxide (TMAOH), tetramethyl ammonium bromide, and tetrapropyl ammonium bromide. The base can include transition metal oxides and hydroxides, alkali metal oxides and hydroxides, alkaline earth metal oxides and hydroxides. For example, the base can include sodium hydroxide or potassium hydroxide. The first solvent can include water.

In some embodiments, the first mixture can comprise water, sodium hydroxide, colloidal silica, tetramethyl ammonium hydroxide, aluminum isopropoxide, and tetramethylammonium bromide. In some embodiments, the first mixture can comprise water, sodium hydroxide, tetraethylorthosilane, and tetrapropyl ammonium hydroxide. In some embodiments, the first mixture can comprise water, tetraethylorthosilane, sodium hydroxide, tetramethyl ammonium hydroxide, and aluminum isopropoxide. In some embodiments, the first mixture can comprise water, sodium hydroxide, tetrapropyl ammonium hydroxide, silicon, and ethanol.

The amount of silicon source present in the first mixture can be from 1.7 mol % to 5.2 mol % (e.g., from 3.1 mol % to 3.8 mol %) of the components used to form the first mixture. The amount of aluminum source present in the first mixture can be from 0.01 mol % to 2 mol % (e.g., from 0.02 mol % to 1 mol %) of the components used to form the first mixture. The amount of organic agent present in the first mixture can be from 0.1 mol % to 5 mol % (e.g., from 0.6 mol % to 0.3 mol %) of the components used to form the first mixture. The amount of base present in the first mixture can be from 0.001 mol % to 0.1% mol % (e.g., from 0.0001 mol % to 0.05 mol %) of the components used to form the first mixture. The amount of solvent present in the mixture can be from 90 mol % to 99 mol % (e.g., from 95 mol % to 99 mol %) of the components used to form the first mixture.

In an exemplary method, the silicon source, aluminum source, base, organic agent, and solvent can be combined in a suitable ratio to form a first mixture comprising 0.048 Na₂O:2.40 (TMA)₂O(2OH):1.2 (TMA)₂O(2Br): 4.35 SiO₂:1.0 Al₂O₃:249 H₂O, after hydrolysis.

In another exemplary method of preparing zeolite nanoparticles, Ludox HS-30 and tetramethylammonium hydroxide can be mixed at room temperature to produce a silicon source. Aluminum isopropoxide can be dissolved in water and tetramethylammonium hydroxide. The resulting mixture can be heated followed by addition of tetramethylammonium bromide, thereby forming the aluminum source. The silicon source and aluminum source can be mixed and aged at room temperature with stirring for about three days. The aged mixture can be heated with stirring for about four days. The reacted mixture can be centrifuged to produce zeolite Y nanoparticles and a supernatant. The supernatant can be mixed with sodium hydroxide, aged overnight, and refluxed for about 3 hours to produce a second batch of zeolite Y nanoparticles and a second supernatant. The second batch of zeolite Y nanoparticles can be separated from the supernatant. The addition of sodium hydroxide, aging, heating, and separating the nanoparticles from the supernatant can define one cycle. The cycle can then be repeated eight times.

In a further exemplary method of preparing zeolite nanoparticles, Ludox HS-30 and tetramethylammonium hydroxide can be mixed at room temperature to produce a silicon source. Aluminum isopropoxide can be dissolved in water and tetramethylammonium hydroxide. The resulting mixture can be heated to form a solution followed by addition of tetramethylammonium bromide resulting in the aluminum source. The silicon source and aluminum source can be mixed and aged at room temperature with stirring for about three days. The aged mixture can be heated with stirring for about four days. The reacted mixture can be centrifuged to produce zeolite Y nanoparticles and a supernatant. The supernatant can be mixed with sodium hydroxide, refluxed, and concentrated by removing water (by condensation) for about 30 minutes during reflux. The resulting concentrated solution can be refluxed for an additional 30 minutes. Sodium hydroxide can be dissolved in the condensed water which can be used to dilute the concentrated solution. The water can be added to the concentrated solution over about 30 minutes. The 90 minutes process can define one cycle. The cycle can be repeated for six times (9 hours) to form zeolite Y nanoparticles.

Methods of incorporating silver in the zeolite nanoparticles are describes in J. Phys. Chem. C 2014, 118, 28580-28591, which is incorporated by reference herein. Briefly, a colloidal dispersion of the zeolite nanoparticles can be ion exchanged first with a sodium salt, such as sodium nitrate and then with a silver salt, such as silver nitrate to form zeolite nanoparticles comprising silver ions. The silver ions in the zeolite nanoparticles can be reduced to form silver nanoparticles. In particular, the silver-exchanged zeolite dispersion formed can be reduced using a reducing agent. Preferably, the reducing agent is a weak reducing agent such as resorcinol. By removing the reducing agent at any stage of the reduction, stable silver nanoparticles on zeolite nanoparticles can be isolated. The properties of the zeolite nanoparticles comprising silver can be characterized with optical spectroscopy (e.g. surface-enhanced Raman measurements) and transmission electron microscopy.

Methods of Using

Also disclosed herein are methods for using the antimicrobial compositions. The antimicrobial compositions can be used to kill or inhibit the growth of a microbe. The methods of killing or inhibiting the growth of a microbe can comprise exposing the microbe to a composition comprising zeolite nanoparticles, wherein the zeolite nanoparticles comprise an effective amount of silver to kill or inhibit the growth of the microbe. As used herein, “inhibit” or other forms of the word, such as “inhibiting” or “inhibition,” refers to lowering of an event or characteristic (e.g., microbe population/infection). It is understood that the inhibition is typically in relation to some standard or expected value. For example, “inhibiting the growth of microbes” means reducing the growth of a microbe relative to a standard or a control.

The antimicrobial compositions can also be used to treat or prevent a microbial infection in a subject. The methods for treating or preventing a microbial infection in a subject can comprise administering a composition comprising the subject zeolite nanoparticles to the patient, wherein the zeolite nanoparticles comprise a therapeutically effective amount of silver. As used herein, “prevent” or other forms of the word, such as “preventing” or “prevention,” refers to stopping a particular event or characteristic, stabilizing or delaying the development or progression of a particular event or characteristic, or minimizing the chances that a particular event or characteristic will occur. “Prevent” does not require comparison to a control as it is typically more absolute than, for example, “reduce.” As used herein, something could be reduced but not prevented, but something that is reduced could also be prevented. Likewise, something could be prevented but not reduced, but something that is prevented could also be reduced. As used herein, “treat” or other forms of the word, such as “treated” or “treatment,” refers to administration of a composition or performing a method in order to reduce, prevent, inhibit, or eliminate a particular characteristic or event (e.g., microbe growth or survival). The term “control” is used synonymously with the term “treat.”

As used herein, by a “subject” is meant an individual. The “subject” can include a mammal, such as a primate or a human. In some embodiments, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds.

As described herein, the term “microbes” includes, for example, bacteria, virus, algae, and fungi. In some embodiments, the methods described herein can be used to kill, inhibit, control or prevent microbes such as Escherichia coli, Staphylococcus aureus, Bacillus coagulans, Bacillus megaterium, Bacillus subtilis, Enterococcus faecium, Pseudoxanthomonas spp., Pseudomonas putida, Pseudomonas aeruginosa, Pseudomonas maculicola, Pseudomanas chlororaphis, Pseudomonas flourescens, Nocardia brasiliensis, Nocardia globerula, Acinetobacter genomospecies, Acinetobacter calcoaceticus, Acinetobacter baumannii, Stenotrophomonas maltophlia, Pantoea stewartii ss stewartii, Chryseobacterium balustinus, Duganella zoogloeoides, Chryseobacterium meningosepticum, Staphylococcus hominis, Nocardia transvalensis, Burkolderia glumea, Pediococcus acidilactici/parvulus, Sphingomonas terrae, Corynebacterium spp., Gordonia rubripertincta, Rhodococcus rhodnii, Brevundimonas vesicularis, Providencian heimbachae, Gordonia sputi, Cellulosimicrobium cellulans, Sphingomonas sanguinis, Hydrogenophaga pseudoflava, Actinomadura cremea, Xanthomonas spp., Candida albicans, Candida parapsilosis, Candida tropicalis, Candida glabrata, Kluyveromyces marxianus, Hyphopichia burtanii, Fusarium oxysporum, Botrytis cinerea, Aspergillus niger, Alternaria alternata, Sclerotinia sclerotiorum, Paecilomyces lilacinus, Penicillium vinaceum, Penicillium expansum, Penicillium charlesii, Penicillium expansum, or a combination thereof.

The methods described herein are useful in treating a variety of microbial infections, including drug-resistant microbial infections and biofilm-associated infections. For example, the methods described herein are useful in treating a variety of infections due to Escherichia coli or Staphylococcus. In some examples, the methods can be used to kill, inhibit, or prevent Gram positive bacteria.

The activity of the antimicrobial agents can be measured in standard assays, e.g., HPLC assays. The compositions can be evaluated for antibacterial activity using the Mueller Hinton (MH) broth antibacterial assay as specified by the Clinical and Laboratory Standards Institute MIC broth microdilution protocol (see Methods for Dilution Antimicrobial Susceptibility Tests for Bacteria That Grow Aerobically; Approved Standard, In The Clinical and Laboratory Standards Institute (CLSI, formerly NCCLS), 7^{th} ed., January 2006, 26 (2), M7-A7; see also Performance Standards for Antimicrobial Susceptibility Testing; Eighteenth Informational Supplement, In The Clinical and Laboratory Standards Institute (CLSI, formerly NCCLS), January 2008, 28 (1), M100-S18.

In some examples, the methods described herein can result in a reduction in the population of microbes of 5 log or more (e.g., 5.5 log or more, 6 log or more, 6.5 log or more, or 7 log or more). In some examples, the methods described herein can result in a reduction in the population of microbes of 5 log or more in 30 seconds (e.g., population of microbes exposed to the composition for 30 seconds). In some examples, the methods described herein can result in complete (100%) reduction in the population of microbes.

The methods and compositions as described herein are useful for both prophylactic and therapeutic treatment. For prophylactic use, a therapeutically effective amount of the compositions described herein are administered to a subject prior to onset (e.g., before obvious signs of a microbial infection), during early onset (e.g., upon initial signs and symptoms of a microbial infection), or after an established inflammatory response or development of a microbial infection. Prophylactic administration can occur for several days to years prior to the manifestation of symptoms of an infection. Therapeutic treatment involves administering to a subject a therapeutically effective amount of the compositions described herein after a microbial infection is diagnosed.

The methods and compositions as described herein are useful in food container coatings. In some embodiments, the compositions can be formulated with coat a food container. In some embodiments, the composition forms a continuous barrier coating on the food or food container. The container can be a glass container, a metal container, a plastic container or a paper container (e.g., a waxed paper container).

The methods and compositions as described herein are useful for medical devices and wound dressing coatings. The medical devices or bandages can be wholly or partially coated with a composition as described herein. In some embodiments, the compositions can be formulated with a wound dressing, coated on a bandage or the exterior surface of a medical device. Exemplary medical devices can include, suture thread, wound closure tape, catheters, tubes, stents, atheroscopic balloons, pace makers, replacement joints (e.g., hip, knee), valves, chips (e.g., information storage media, computer chip, computer-readable media), etc.

The methods and compositions as described herein are also useful in other coatings such as wall coatings (e.g., paints, varnishes, etc.).

By way of non-limiting illustration, examples of certain embodiments of the present disclosure are given below.

### EXAMPLES

### Example 1: Sunscreen Formulations

Zeolite nanoparticles having a faujasite structure were prepared. The zeolite particles had an average particle size of from 30-150 nm.

A sample of sunscreen agent comprising avobenzone encapsulated in the zeolite nanoparticles was then prepared. To encapsulate the avobenzone, the zeolite nanoparticles were first calcined at 400° C. for six hours. The nanoparticles were then exposed to a solution of avobenzone in methanol, air dried, and sonicated to break up any agglomerates. A sample of sunscreen agent comprising avobenzone encapsulated in a zeolite nanoparticles that include a quenching ion. These sunscreen agents were prepared as above, except that thallium was introduced into the zeolite particles by ion exchange. The avobenzone loading in the zeolite nanoparticles was estimated to be 14% by weight, based on the total weight of the sunscreen agent.

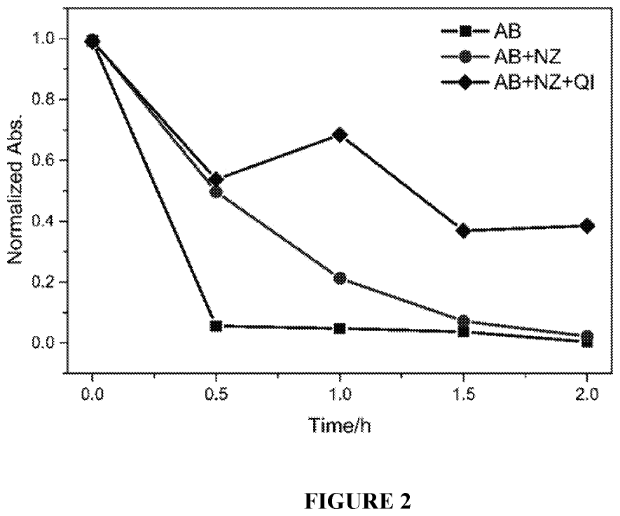

UV/Visible spectrometry was used to assess the photostability of the sunscreen agents prepared above. FIG. 2 is a plot illustrating the normalized absorbance of each sample (avobenzone alone, square trace; avobenzone encapsulated in a zeolite nanoparticle, circle trace; and avobenzone encapsulated in a zeolite nanoparticle that includes a quenching ion, diamond trace) as a function of time. Encapsulation of avobenzone in the zeolite nanoparticle stabilizes the avobenzone to degradation. All samples were prepared by depositing a methanol solution of the various compounds on a quartz plate and then evaporation of the solvent

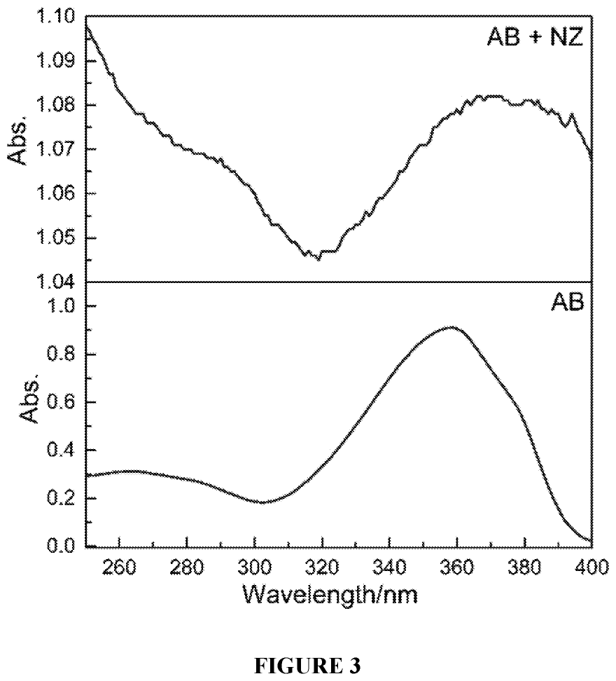

FIG. 3 illustrates a UV/Vis absorbance spectrum of avobenzone alone in methanol (bottom trace) compared with a UV/Vis absorbance spectrum of avobenzone encapsulated in zeolite nanoparticles dried on a quartz plate from a methanolic solution (top trace). Upon encapsulation of the avobenzone in the zeolite nanoparticle, significant scattering is observed, along with a broadening and bathochromic shift in absorption (>400 nm).

Subsequently, a second-generation sunscreen formulation was prepared using a sunscreen agent comprising avobenzone encapsulated in faujasite zeolite nanoparticles using cyclohexane. By using cyclohexane instead of methanol, the loading of avobenzone in the zeolite nanoparticles could be significantly increased. A composition for use was prepared by dispersing this sunscreen agent in petroleum jelly.

A UV photolysis lamp having a light flux of 320 mW equipped with a cutoff filter at 270-280 nm was used to evaluate the performance and stability of the sunscreen compositions prepared herein. With 3 min of exposure to the UV lamp (same position as sample, after the filter), a clear damage was observed on human skin. Typically, under hot sun, this sunburn process takes 30 min to an hour. Accordingly, the photolysis lamp used for analysis is considerably more intense than natural sunlight.

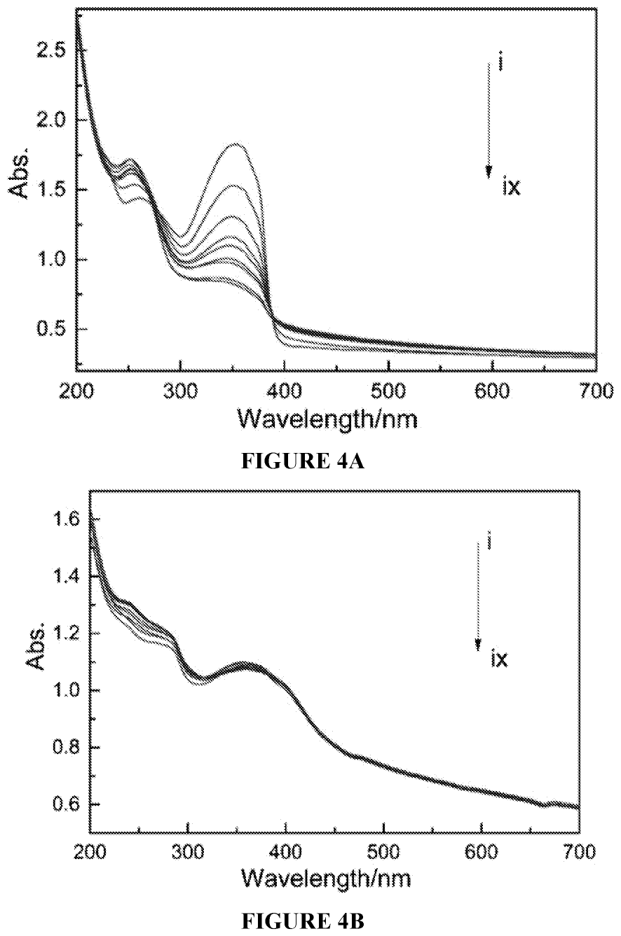

FIGS. 4A and 4B compare the stability of avobenzone dispersed in petroleum jelly with avobenzone encapsulated in encapsulated in zeolite nanoparticles (10% zeolite, 1% AB) dispersed in petroleum jelly. As shown by comparison of FIGS. 4A and 4B, avobenzone is significantly stabilized through encapsulation in the pores of the nanozeolite particles. This formulation was found to be exceedingly stable when stored a dry environment.

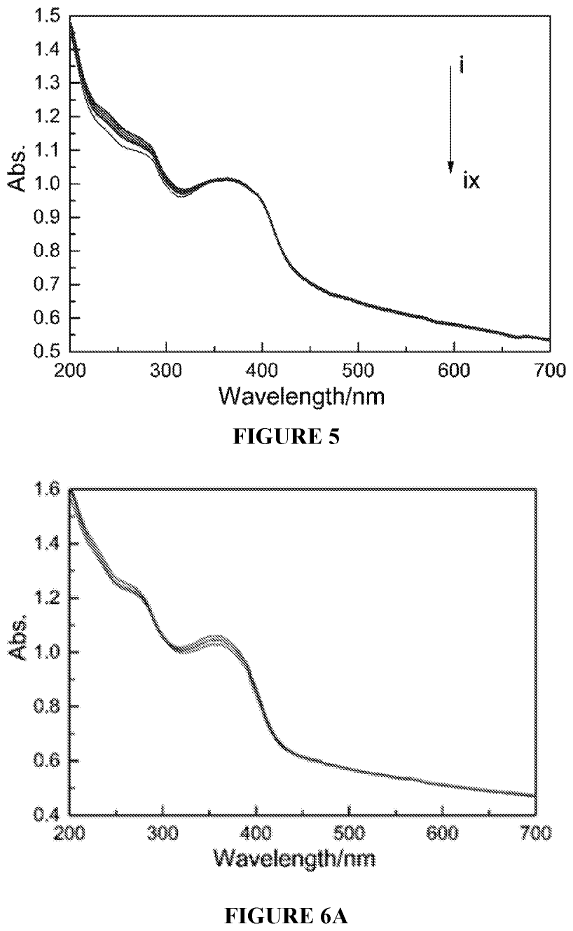

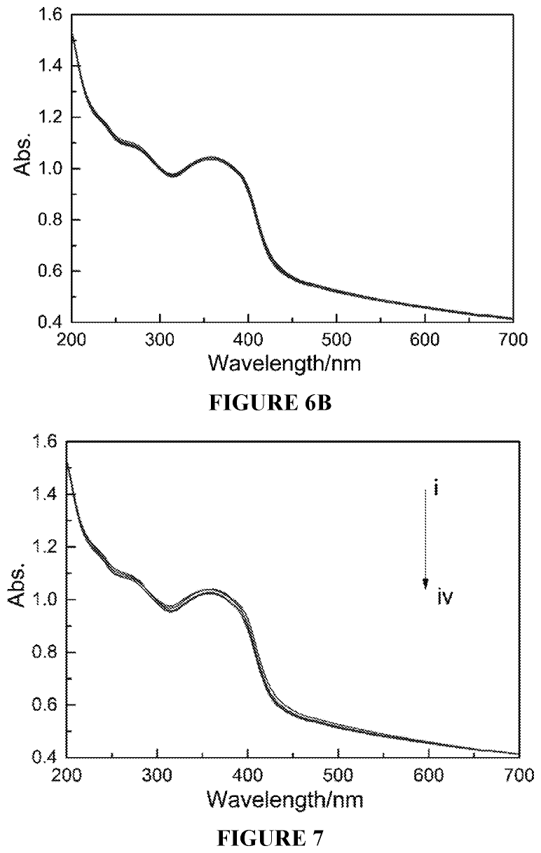

In a subsequent example, the surface of the zeolite nanoparticles was covalently modified with hexadecylamine (HDA) to render the sunscreen agent hydrophobic so that it disperses better in petroleum jelly. The contact angle of HDA-modified zeolite was 72.5°. As shown in FIG. 5, avobenzone encapsulated within the HDA-modified zeolite also exhibited improved stability upon irradiation with UV light. As shown in FIGS. 6A and 6B, this formulation was found to be stable to photolysis after storage in a dry environment for a period of at least 28 days.

FIG. 7 is a plot showing the long-term stability of avobenzone/nanozeolite formulations. The absorption spectra of a thin film of avobenzone encapsulated in zeolite nanoparticles (10% zeolite, 1% AB) dispersed in petroleum jelly on quartz plates was obtained following 0, 3, 5, and 12 hours of irradiation using a UV photolysis lamp. As shown in FIG. 7, little to no degradation of the avobenzone was observed via UV spectroscopy. In contrast, in the case of a formulation containing only avobenzone, complete degradation of the avobenzone was observed within four hours.

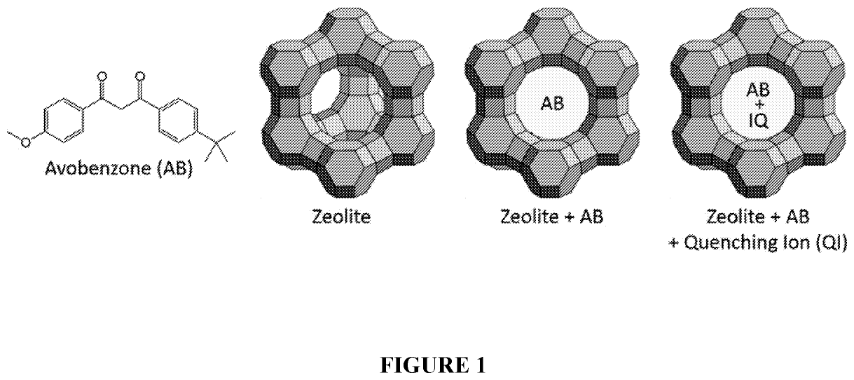

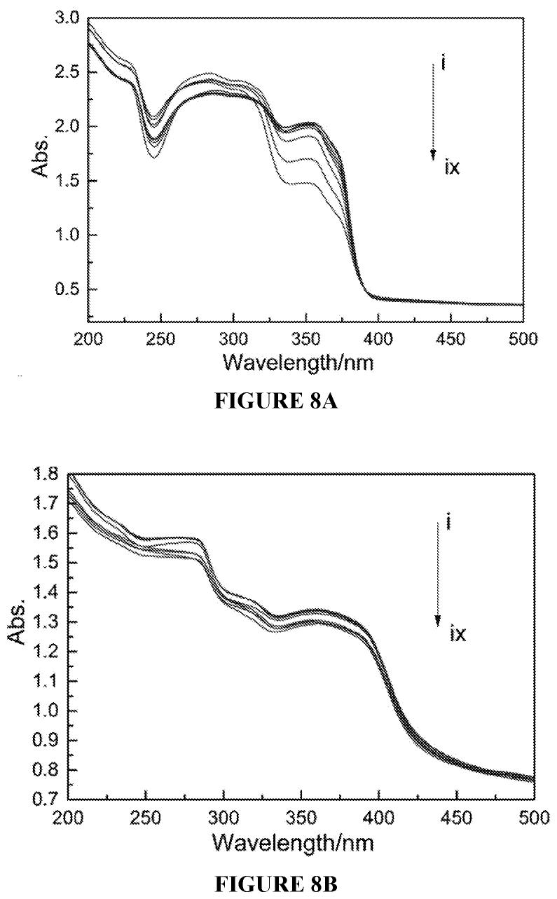

FIGS. 8A and 8B show the spectra in the photolysis process for 4 hours of 1% OMC (octinoxate) and 1% AB in petroleum jelly (FIG. 8A) and 1% OMC and (10% zeolite, 1% AB) HDA-ABNZ in petroleum jelly (FIG. 8B). With both AB and OMC as free molecules in petroleum jelly, AB decomposition was observed after the first hour of photolysis. By encapsulating AB in zeolite, AB and OMC are not accessible for reaction with each other, and the decomposition process is inhibited.

### Example 2: Antimicrobial Formulations

Zeolite nanoparticles having a faujasite structure were prepared. The zeolite particles had an average particle size of from 30-150 nm.

The zeolite nanoparticles were then ion-exchanged with 0.01 M AgNO₃ to replace supercage Na⁺ cations with Ag⁺. Silver nanoparticles were deposited on the nanozeolites by reduction with resorcinol, with samples recovered after 1, 3, and 5 h which afforded silver nanoparticles of 1-2 nm, 2-3 nm, and 3-5 nm size. The size dimensions of the silver nanoparticles were measured using HRTEM. The silver loading of the nanozeolites was verified with atomic absorption.

Preliminary qualitative experiments demonstrated that the silver-loaded nanozeolites (Ag-NZ) dramatically reduced bacterial colony formation. Even at the lowest doses initially screened (50 μg/mL), a dramatic reduction in colony numbers was observed. Further quantitative assessment of the antimicrobial activity of the compositions will be performed as described below.

Assay of Antimicrobial Activity:

Two modes of antimicrobial activity will be evaluated—bacteriostatic activity (inhibition of bacterial cell proliferation) and bactericidal activity (killing of bacteria). Two species of bacteria will be tested—Escherichia coli (E. coli) as a representative gram-negative specie, and Staphylococcus aureus (S. aureus) as a representative gram-positive. Gram-positive bacteria have thicker cell walls (˜10 nm) that contain three to twenty times more peptidoglycan than gram-negative bacteria (2-3 nm). E. coli K12 will be cultured in standard LB broth and S. aureus USA 300 will be cultured in brain-heart infusion broth. Bacteria will be propagated by shaking at 225 RPM overnight at 37° C. prior to set-up for experiments.

Assay of Bacteriostatic Activity:

determination of minimum inhibitory concentration (MIC): Bacteria concentrations will be adjusted to obtain an optical density of −0.2 and added to 96-well U-bottom culture plates, 90 μL/well. AgNP-NZ at various concentrations in a total volume of 10 μL will be added to bacteria to attain final Ag concentrations in wells of 0, 10 ng/mL, 100 ng/mL, 1 μg/mL, 10 μg/mL, and 100 μg/mL, in a total volume of 100 μL/well, 4 wells per Ag concentration. Controls will include wells containing bacteria-free culture medium and medium plus AgNP-NZ. Plates will be shaken at 37° C. and optical densities (OD) of wells will be measured at 30 minute intervals from time 0 through 180 minutes with a SpectraMax 190 microplate reader. Mean ODs and standard deviations for each Ag concentration will be calculated from quadruplicate wells. Statistical significance will be determined by Student's t test. Minimal inhibitory concentration (MIC) of Ag will be taken as the lowest concentration of Ag that inhibits bacterial proliferation as indicated by OD.

Assay of Bactericidal Activity; Time-Kill Test:

Bacteria concentrations will be adjusted to 5×10⁵ colony-forming units (cfu)/mL and suspended in their respective media in sterile glass tubes, 1.8 mL/tube. AgNP-NZ at various concentrations in a total of 200 μL will be added to bacteria to attain final Ag concentrations in tubes of the MIC, 0.1 MIC, and 0.01 MIC (determined in bacteriostatic assay above) in a total volume of 2.0 mL/tube, 3 tubes per Ag concentration. Control tubes will contain bacteria but no Ag. Tubes will be shaken at 37° C. and 100 μL samples will be withdrawn from each tube at time 0, 0.5, 1.0, 2.0, 4.0, 6.0, 8.0, 10.0, 12.0, and 24.0 hours. Bacteria will be isolated from AgNP-NZ by centrifugation (400×g, 10 min) and the bacterial pellets will be rinsed and suspended in a series of five 10-fold dilutions of appropriate culture medium. Aliquots of each sample will be spread upon 100 mm petri plates containing the appropriate agarose-supplemented culture medium, 100 μL of sample per plate, 3 replicate plates per sample. Plates will be incubated overnight at 37° C. and resulting bacterial colonies will be counted. Mean colony forming units (cfu) and standard deviations will be calculated from triplicate wells and used to define the minimum bactericidal concentration (MBC) and to plot a time-kill curve.

### Example 3: Controlled Release Insecticide Formulations

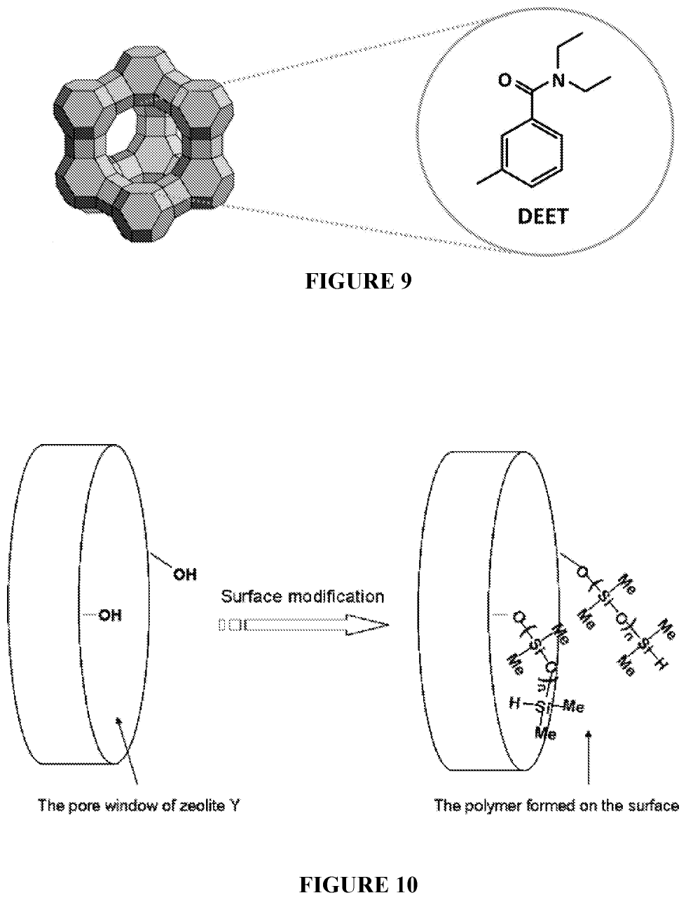

Insecticide formulations will be prepared by encapsulating DEET (N,N-diethyl-3-methylbenzamide) in zeolite nanoparticles. As shown in FIG. 9, DEET can be encapsulated within the pores of the zeolite nanoparticles. Thus, while resident on the skin of the subject, the DEET molecules will largely be encapsulated within the zeolites, sequestered from interactions with the immune system of the subject. Therefore, any allergic response associated with DEET can be minimized. The zeolite nanoparticles will also provide for the extended release of DEET. In this way, the insecticide formulations can provide a controlled release of DEET over an extended period of time while minimizing contact between the DEET and the subject's skin.

The compositions of the appended claims are not limited in scope by the specific compositions described herein, which are intended as illustrations of a few aspects of the claims. Any compositions that are functionally equivalent are intended to fall within the scope of the claims. Various modifications of the compositions in addition to those shown and described herein are intended to fall within the scope of the appended claims. Further, while only certain representative compositions disclosed herein are specifically described, other combinations of the components described herein also are intended to fall within the scope of the appended claims, even if not specifically recited. Thus, a combination of components or constituents may be explicitly mentioned herein or less, however, other combinations of components and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference. Unless otherwise specifically described, all percentages included herein are percentages by weight, based on total weight of the composition, excluding any propellant that may be present.

What is claimed is:

1. A composition comprising zeolite nanoparticles dispersed in a topically acceptable carrier, wherein the zeolite nanoparticles further comprise an effective amount of an active agent adsorbed on the zeolite nanoparticles, encapsulated within the zeolite nanoparticles, or a combination thereof.

2. The composition of claim 1, wherein the zeolite nanoparticles have an average particle size of less than 250 nm.

3. The composition of any of claims 1-2, wherein the zeolite nanoparticles have an average particle size of less than 100 nm.

4. The composition of any of claims 1-3, wherein the zeolite nanoparticles have an average particle size of from 10 to 100 nm.

5. The composition of any of claims 1-4, wherein the zeolite nanoparticles have an average particle size of from 10 to 50 nm.

6. The composition of any of claims 1-5, wherein the zeolite nanoparticles exhibit an internal surface area of from 100 to 1,000 m²/g.

7. The composition of any of claims 1-6, wherein the zeolite nanoparticles exhibit an internal surface area of from 200 to 1,000 m²/g.

8. The composition of any of claims 1-7, wherein the zeolite nanoparticles comprise a faujasite structure.

9. The composition of any of claims 1-8, wherein the active agent is chosen from UV-blocking agents, antimicrobial agents, insecticides, cosmetic agents, fragrances, anesthetic agents, keratolytic agents, steroids, anthelmintic agents, dermatological agents, antioxidants, anti-inflammatory agents, and combinations thereof.

10. The composition of claim 9, wherein the active agent is an insecticide.

11. The composition of claim 10, wherein the insecticide comprises N,N-diethyl-meta-toluamide (DEET).

12. The composition of any of claims 1-11, wherein the active agent comprises metal nanoparticles.

13. The composition of any of claims 1-12, wherein the active agent comprises metal ions.

14. The composition of any of claims 1-13, wherein the active agent comprises a small molecule.

15. The composition of claim 1-14, wherein the active agent comprises a hydrophilic small molecule.

16. The composition of any of claims 1-14, wherein the active agent comprises a hydrophobic small molecule.

17. The composition of any of claims 1-16, wherein the active agent has a molecular size of 13 Angstroms or less.

18. The composition of any of claims 1-17, wherein the active agent comprises a charged small molecule.

19. The composition of any of claims 1-18, wherein the active agent comprises a neutral small molecule.

20. The composition of any of claims 1-19, wherein the active agent is encapsulated within the zeolite nanoparticles.

21. The composition of any of claims 1-19, wherein the zeolite nanoparticles comprise a hydrophobic surface.

22. The composition of any of claims 1-19, wherein the zeolite nanoparticles comprise a hydrophilic surface.

23. The composition of any of claims 1-22, wherein the zeolite nanoparticles comprise a charged surface.

24. The composition of any of claims 1-22, wherein the zeolite nanoparticles comprise a neutral surface.

25. The composition of any of claims 1-24, wherein the active agent is present in an amount of from 1% to 25% by weight, based on the total weight of the zeolite nanoparticles.

26. The composition of any of claims 1-25, wherein the active agent is present in an amount of from 5% to 20% by weight, based on the total weight of the zeolite nanoparticles.

27. The composition of any of claims 1-26, wherein in the composition is a sunscreen or cosmetic.

28. The composition of any of claims 1-27, wherein the composition is a cream, dispersion, emulsion, gel, ointment, lotion, milk, mousse, spray, or tonic.

29. The composition of any of claims 1-28, wherein the active agent is encapsulated within the zeolite nanoparticles, and wherein the active agent remains encapsulated within the zeolite nanoparticles upon application of the composition to a subject's skin.

30. The composition of claim 29, wherein the active agent is stable towards degradation for a period of at least eight hours upon application of the composition to the subject's skin.

31. The composition of any of claims 1-30, wherein the active agent is encapsulated within the zeolite nanoparticles, and wherein the active agent is stable towards degradation for a period of at least six months when stored at room temperature in the absence of light.

32. The composition of any of claims 1-31, wherein the active agent is adsorbed on the zeolite nanoparticles, encapsulated within the zeolite nanoparticles, or a combination thereof, and wherein the active agent is released from the zeolite nanoparticles upon application of the composition to a subject's skin.

33. The composition of claim 32, wherein the active agent is released over an extended period of time.

34. The composition of claim 33, wherein the active agent is released over a period of at least four hours.

35. A composition comprising a sunscreen agent dispersed in a topically acceptable carrier, wherein the sunscreen agent comprises an organic UV-blocking agent encapsulated within a porous inorganic nanomaterial.

36. The composition of claim 35, wherein the UV-blocking agent exhibits a molar extinction coefficient of at least 10,000 mol⁻¹ L cm⁻¹ for at least one wavelength within the range of from 290 nm to 400 nm.

37. The composition of any of claims 35-36, wherein the UV-blocking agent is chosen from p-aminobenzoic acid, padiate O, phenylbenzimidazole sulfonic acid, cinoxate, dixoybenzone, oxybenzone, homosalate, menthyl anthranilate, octocrylene, octyl methoxycinnamate, octyl salicylate, sulisobenzone, trolamine salicylate, avobenzone, ecamsule, 4-methylbenzylidene camphor, bisoctrizole, bemotrizinol, bisdisulizole disodium, tris-biphenyl triazine, drometrizole trisiloxane, benzophenone-9, ethylhexyl triazone, diethylamino hydroxybenzoyl hexyl benzoate, iscotrizinol, polysilicone-15, amiloxate, and combinations thereof.

38. The composition of any of claims 35-37, wherein the UV-blocking agent is chosen from p-aminobenzoic acid, padiate O, phenylbenzimidazole sulfonic acid, cinoxate, dixoybenzone, oxybenzone, homosalate, menthyl anthranilate, octocrylene, octyl methoxycinnamate, octyl salicylate, sulisobenzone, trolamine salicylate, avobenzone, ecamsule, and combinations thereof.

39. The composition of any of claims 35-38, wherein the UV-blocking agent is chosen from avobenzone, oxybenzone, and combinations thereof.

40. The composition of any of claims 35-39, wherein the porous inorganic nanomaterial exhibits a monodisperse pore size distribution.

41. The composition of any of claims 35-40, wherein the porous inorganic nanomaterial exhibits a pore size of from 10 to 75 angstroms.

42. The composition of any of claims 35-41, wherein the porous inorganic nanomaterial exhibits a pore size of from 10 to 50 angstroms.

43. The composition of any of claims 35-42, wherein the porous inorganic nanomaterial is capable of scattering UV light.

44. The composition of any of claims 35-43, wherein the porous inorganic nanomaterial has an average particle size of less than 250 nm.

45. The composition of any of claims 35-44, wherein the porous inorganic nanomaterial has an average particle size of less than 100 nm.

46. The composition of any of claims 35-45, wherein the porous inorganic nanomaterial has an average particle size of from 10 to 100 nm.

47. The composition of any of claims 35-46, wherein the porous inorganic nanomaterial has an average particle size of from 10 to 50 nm.

48. The composition of any of claims 35-47, wherein the porous inorganic nanomaterial exhibits an internal surface area of from 100 to 1,000 m²/g.

49. The composition of any of claims 35-48, wherein the porous inorganic nanomaterial exhibits an internal surface area of from 200 to 1,000 m²/g.

50. The composition of any of claims 35-49, wherein the porous inorganic nanomaterial comprises an alumino-silicate nanoparticle.

51. The composition of claim 50, wherein the alumino-silicate nanoparticle comprises a zeolite nanoparticle.

52. The composition of claim 51, wherein the zeolite nanoparticle comprises a faujasite structure.

53. The composition of any of claims 35-49, wherein the porous inorganic nanomaterial comprises a metal-organic framework.

54. The composition of claim 53, wherein the metal-organic framework is chosen from an iron(III) dicarboxylate, an iron(III) tetramethylterephthalate, an iron(III) muconate, a zinc terephthalate, a zinc imidazolate, and combinations thereof.

55. The composition of claim 53, wherein the metal-organic framework is chosen from MIL-88A, MIL-88B-4CH3, MIL-89, MIL-100(Fe), MIL-53(Fe), MOF-5, ZIF-8, and combinations thereof.

56. The composition of any of claims 35-55, wherein the sunscreen agent comprises from 5% to 20% by weight UV-blocking agent, based on the total weight of the sunscreen agent.

57. The composition of any of claims 35-56, wherein the composition has an SPF of at least 15.

58. The composition of any of claims 35-57, wherein the composition exhibits a monochromatic protection factor of greater than or equal to 3 in 340-400 nm range and a critical wavelength of greater than or equal to 370 nm.

59. The composition of any of claims 35-58, wherein the sunscreen agent is present in an amount of from 0.5% to 30% by weight, based on the total weight of the composition.

60. The composition of any of claims 35-59, wherein in the composition is a sunscreen or cosmetic.

61. The composition of any of claims 35-60, wherein the composition is a cream, dispersion, emulsion, gel, ointment, lotion, milk, mousse, spray, or tonic.

62. The composition of any of claims 35-61, wherein the porous inorganic nanomaterial comprises a hydrophobic surface.

63. A composition comprising sunscreen agent dispersed in a topically acceptable carrier, wherein the sunscreen agent comprises an organic UV-blocking agent encapsulated within a porous inorganic nanomaterial, and
wherein the porous inorganic nanomaterial has an average particle size of less than 250 nm.

64. The composition of claim 63, wherein the UV-blocking agent exhibits a molar extinction coefficient of at least 10,000 mol⁻¹ L cm⁻¹ for at least one wavelength within the range of from 290 nm to 400 nm.

65. The composition of any of claims 63-64, wherein the UV-blocking agent is chosen from p-aminobenzoic acid, padiate O, phenylbenzimidazole sulfonic acid, cinoxate, dixoybenzone, oxybenzone, homosalate, menthyl anthranilate, octocrylene, octyl methoxycinnamate, octyl salicylate, sulisobenzone, trolamine salicylate, avobenzone, ecamsule, 4-methylbenzylidene camphor, bisoctrizole, bemotrizinol, bisdisulizole disodium, tris-biphenyl triazine, drometrizole trisiloxane, benzophenone-9, ethylhexyl triazone, diethylamino hydroxybenzoyl hexyl benzoate, iscotrizinol, polysilicone-15, amiloxate, and combinations thereof.

66. The composition of any of claims 63-65, wherein the UV-blocking agent is chosen from p-aminobenzoic acid, padiate O, phenylbenzimidazole sulfonic acid, cinoxate, dixoybenzone, oxybenzone, homosalate, menthyl anthranilate, octocrylene, octyl methoxycinnamate, octyl salicylate, sulisobenzone, trolamine salicylate, avobenzone, ecamsule, and combinations thereof.

67. The composition of any of claims 63-66, wherein the UV-blocking agent is chosen from avobenzone, oxybenzone, and combinations thereof.

68. The composition of any of claims 63-67, wherein the porous inorganic nanomaterial exhibits a monodisperse pore size distribution.

69. The composition of any of claims 63-68, wherein the porous inorganic nanomaterial exhibits a pore size of from 10 to 75 angstroms.

70. The composition of any of claims 63-69, wherein the porous inorganic nanomaterial exhibits a pore size of from 10 to 50 angstroms.

71. The composition of any of claims 63-70, wherein the porous inorganic nanomaterial is capable of scattering UV light.

72. The composition of any of claims 63-71, wherein the porous inorganic nanomaterial has an average particle size of less than 100 nm.

73. The composition of any of claims 63-72, wherein the porous inorganic nanomaterial has an average particle size of from 10 to 100 nm.

74. The composition of any of claims 63-73, wherein the porous inorganic nanomaterial has an average particle size of from 10 to 50 nm.

75. The composition of any of claims 63-74, wherein the porous inorganic nanomaterial exhibits an internal surface area of from 100 to 1,000 m²/g.

76. The composition of any of claims 63-75, wherein the porous inorganic nanomaterial exhibits an internal surface area of from 200 to 1,000 m²/g.

77. The composition of any of claims 63-76, wherein the porous inorganic nanomaterial comprises an alumino-silicate nanoparticle.

78. The composition of claim 77, wherein the alumino-silicate nanoparticle comprises a zeolite nanoparticle.

79. The composition of claim 78, wherein the zeolite nanoparticle comprises a faujasite structure.

80. The composition of any of claims 63-79, wherein the porous inorganic nanomaterial comprises a metal-organic framework.

81. The composition of claim 80, wherein the metal-organic framework is chosen from an iron(III) dicarboxylate, an iron(III) tetramethylterephthalate, an iron(III) muconate, a zinc terephthalate, a zinc imidazolate, and combinations thereof.

82. The composition of claim 80, wherein the metal-organic framework is chosen from MIL-88A, MIL-88B-4CH3, MIL-89, MIL-100(Fe), MIL-53(Fe), MOF-5, ZIF-8, and combinations thereof.

83. The composition of any of claims 63-82, wherein the sunscreen agent comprises from 5% to 20% by weight UV-blocking agent, based on the total weight of the sunscreen agent.

84. The composition of any of claims 63-83, wherein the composition has an SPF of at least 15.

85. The composition of any of claims 63-84, wherein the composition exhibits a monochromatic protection factor of greater than or equal to 3 in 340-400 nm range and a critical wavelength of greater than or equal to 370 nm.

86. The composition of any of claims 63-85, wherein the sunscreen agent is present in an amount of from 0.5% to 30% by weight, based on the total weight of the composition.

87. The composition of any of claims 63-86, wherein in the composition is a sunscreen or cosmetic.

88. The composition of any of claims 63-87, wherein the composition is a cream, dispersion, emulsion, gel, ointment, lotion, milk, mousse, spray, or tonic.

89. The composition of any of claims 63-88, wherein the porous inorganic nanomaterial comprises a hydrophobic surface.

90. A composition comprising zeolite nanoparticles, wherein the zeolite nanoparticles further comprise silver nanoparticles disposed on the zeolite nanoparticles and antimicrobial metal ions retained at ion-exchangeable sites within the zeolite nanoparticles.

91. The composition of claim 90, wherein the antimicrobial metal ions are chosen from silver ions, zinc ions, copper ions, or a combination thereof.

92. The composition of any of claims 90-91, wherein the antimicrobial metal ions are present in an amount of at least 10% of the ion exchange capacity of the zeolite nanoparticles.

93. The composition of any one of claims 90-92, wherein the antimicrobial metal ions are present in an amount of from 10% to 100% of the ion exchange capacity of the zeolite nanoparticles.

94. The composition of any one of claims 90-93, wherein the silver nanoparticles have an average diameter of 10 nm or less.

95. The composition of any one of claims 90-94, wherein the silver nanoparticles have an average diameter of from 1 nm to 10 nm.

96. The composition of any one of claims 90-95, wherein the silver nanoparticles have an average diameter of from 1 nm to 5 nm.

97. The composition of any one of claims 90-96, wherein the silver nanoparticles are present in an amount of at least 1% by weight, based on the total weight of the zeolite nanoparticles and the silver nanoparticles.

98. The composition of any one of claims 90-97, wherein the silver nanoparticles are present in an amount of from 1% to 25% by weight, based on the total weight of the zeolite nanoparticles and the silver nanoparticle.

99. A composition comprising zeolite nanoparticles, wherein the zeolite nanoparticles further comprise silver nanoparticles disposed on the zeolite nanoparticles and wherein a surface of the zeolite nanoparticles is functionalized with a microbial targeting agent.

100. The composition of claim 99, wherein the microbial targeting agent is covalently bound to the surface of the zeolite nanoparticles.

101. The composition of any of claims 99-100, wherein the microbial targeting agent comprises a cationic group or a cationic precursor.

102. The composition of any of claims 99-101, wherein the microbial targeting agent comprises an amine containing group.

103. A composition comprising zeolite nanoparticles, wherein the zeolite nanoparticles further comprise silver nanoparticles disposed on the zeolite nanoparticles and a small molecule antimicrobial agent adsorbed on and/or within the zeolite nanoparticle.

104. The composition of claim 103, wherein the zeolite nanoparticles are present in an amount of from 1% to 25% by weight, based on the total weight of the zeolite nanoparticles and the silver nanoparticle.

105. The composition of any one of claims 103-104, wherein the small molecule antimicrobial agent is present in an amount of from 1% to 20% by weight, based on the total weight of the zeolite nanoparticles and the silver nanoparticle.

106. A method of killing or inhibiting the growth of a microbe, the method comprising: exposing the microbe to a composition comprising zeolite nanoparticles, wherein the zeolite nanoparticles comprise an effective amount of silver to kill or inhibit the growth of the microbe.

107. A method of treating or preventing a microbial infection in a patient, the method comprising: administering a composition comprising zeolite nanoparticles to the patient, wherein the zeolite nanoparticles comprise a therapeutically effective amount of silver.

108. The method of any of claims 106-107, wherein the zeolite nanoparticles have an average diameter of less than 100 nm.

109. The method of claim 108, wherein the average diameter of the zeolite nanoparticles is from 10 nm to less than 100 nm.

110. The method of any of claims 108-109, wherein the average diameter of the zeolite nanoparticles is from 20 nm to 60 nm.

111. The method of any one of claims 106-110, wherein the silver comprises silver nanoparticles.

112. The method of claim 111, wherein the silver nanoparticles have an average diameter of 10 nm or less.

113. The method of any of claims 111-112, wherein the silver nanoparticles have an average diameter of from 1 nm to 10 nm.

114. The method of any one of claims 111-113, wherein the silver nanoparticles have an average diameter of from 1 nm to 5 nm.

115. The method of any one of claims 111-114, wherein the silver nanoparticles are present in an amount of at least 1% by weight, based on the total weight of the zeolite nanoparticles and the silver.

116. The method of any one of claims 111-115, wherein the silver nanoparticles are present in an amount of from 1% to 25% by weight, based on the total weight of the zeolite nanoparticles and the silver.

117. The method of any one of claims 106-116, wherein the silver comprises silver ions retained at ion-exchangeable sites within the zeolite nanoparticles.

118. The method of claim 117, wherein the silver ions are present in an amount of 10% or greater of the ion exchange capacity of the zeolite nanoparticles.

119. The method of any of claims 114-118, wherein the silver ions are present in an amount of from 50% up to 100% of the ion exchange capacity of the zeolite nanoparticles.

120. The method of any one of claims 106-119, wherein the zeolite nanoparticles have an average internal surface area of at least 300 m2/g.

121. The method of any one of claims 106-120, wherein the zeolite nanoparticles further comprise an adjuvant.

122. The method of claim 121, wherein the adjuvant comprises antimicrobial metal ions retained at ion-exchangeable sites within the zeolite nanoparticles.

123. The method of claim 122, wherein the antimicrobial metal ions include copper ions, zinc ions, or a combination thereof.

124. The method of claim 121, wherein the adjuvant includes hydrogen ions.

125. The method of claim 124, wherein the zeolite nanoparticles comprise an effective amount of hydrogen ions to reduce the pH of an aqueous region in contact with the zeolite nanoparticles.

126. The method of claim 121, wherein the adjuvant includes a small molecule antimicrobial agent.

127. The method of claim 126, wherein the small molecule antimicrobial agent is hydrophilic.

128. The method of any one of claims 106-127, wherein the zeolite nanoparticles comprise a microbial targeting agent.

129. The method of claim 128, wherein the microbial targeting agent is covalently bound to a surface of the zeolite nanoparticles.

130. The method of any of claims 128-129, wherein the microbial targeting agent comprises a cationic group or a cationic precursor.

131. The method of any one of claims 106-130, wherein the microbe is selected from a bacteria, a fungi, a virus, an algae, or a combination thereof.

132. The method of claim 131, wherein the microbe is a bacteria selected from Escherichia coli, Staphylococcus aureus, Bacillus coagulans, Bacillus megaterium, Bacillus subtilis, Enterococcus faecium, Pseudoxanthomonas spp., Pseudomonas putida, Pseudomonas aeruginosa, Pseudomonas maculicola, Pseudomanas chlororaphis, Pseudomonas flourescens, Nocardia brasiliensis, Nocardia globerula, Acinetobacter genomospecies, Acinetobacter calcoaceticus, Acinetobacter baumannii, Stenotrophomonas maltophlia, Pantoea stewartii ss stewartii, Chryseobacterium balustinus, Duganella zoogloeoides, Chryseobacterium meningosepticum, Staphylococcus hominis, Nocardia transvalensis, Burkolderia glumea, Pediococcus acidilactici/parvulus, Sphingomonas terrae, Corynebacterium spp., Gordonia rubripertincta, Rhodococcus rhodnii, Brevundimonas vesicularis, Providencian heimbachae, Gordonia sputi, Cellulosimicrobium cellulans, Sphingomonas sanguinis, Hydrogenophaga pseudoflava, Actinomadura cremea, Xanthomonas spp. or a combination thereof.

133. The method of claim 131, wherein the microbe is a fungi selected from Candida albicans, Candida parapsilosis, Candida tropicalis, Candida glabrata, Kluyveromyces marxianus, Hyphopichia burtanii, Fusarium oxysporum, Botrytis cinerea, Aspergillus niger, Alternaria alternata, Sclerotinia sclerotiorum, Paecilomyces lilacinus, Penicillium vinaceum, Penicillium expansum, Penicillium charlesii, Penicillium expansum, or a combination thereof.

134. The method of any one of claims 106-133, wherein the microbe is present on a surface of or in a food product, a wound, a medical device, a pharmaceutical product, a personal care product, an equipment, a wall, a liquid, or a combination thereof.

135. The method of any one of claims 106-134, wherein the composition comprises a powder.

136. The method of any one of claims 106-135, wherein the composition comprises the zeolite nanoparticles dispersed in a carrier.

137. An article comprising zeolite nanoparticles dispersed on a surface of the article, wherein the zeolite nanoparticles comprise an effective amount of silver to kill or inhibit the growth of a microbe.

138. The article of claim 137, wherein the zeolite nanoparticles have an average diameter of less than 100 nm.

139. The article of any of claims 137-138, wherein the average diameter of the zeolite nanoparticles is from 10 nm to less than 100 nm

140. The article of any one of claims 137-139, wherein the silver comprises silver nanoparticles.

141. The article of claim 140, wherein the silver nanoparticles have an average diameter of 10 nm or less.

142. The article of any of claims 140-141, wherein the silver nanoparticles are present in an amount of at least 1% by weight, based on the total weight of the zeolite nanoparticles and the silver.

143. The article of any one of claims 140-142, wherein the silver nanoparticles are present in an amount of from 1% to 25% by weight, based on the total weight of the zeolite nanoparticles and the silver.

144. The article of any one of claims 137-143, wherein the zeolite nanoparticles comprise an adjuvant.

145. The article of claim 144, wherein the adjuvant comprises antimicrobial metal ions retained at ion-exchangeable sites within the zeolite nanoparticles.

146. The article of any of claims 144-145, wherein the antimicrobial metal ions include copper ions, zinc ions, or a combination thereof.

147. The article of claim 144, wherein the adjuvant includes hydrogen ions.

148. The article of claim 147, wherein the zeolite nanoparticles comprise an effective amount of hydrogen ions to reduce the pH of a region in contact with the zeolite nanoparticles.

149. The article of claim 144, wherein the adjuvant includes a small molecule antimicrobial agent.

150. The article of claim 149, wherein the small molecule antimicrobial agent is hydrophilic.

151. The article of any one of claims 137-150, wherein the zeolite nanoparticles comprise a microbial targeting agent.

152. The article of claim 151, wherein the microbial targeting agent comprises a cationic group or a cationic precursor.

153. The article of any of claims 151-152, wherein the microbial targeting agent comprises an amine containing group.

154. The article of any of claims 137-153, wherein the article is a food package, a medical device, or a coating.

155. The composition of any of claims 35-89, wherein the sunscreen agent further comprises a quenching species.
